(19) United States
(12) Patent Application Publication
(10) Pub. No.: US 2011/0064892 A1
(43) Pub. Date: Mar. 17, 2011

(54) Polycyclic Organic Compounds, Retardation Layer and Compensation Panel on Their Base

(75) Inventors: Alexey Nokel, Moscow (RU); Pavel I. Lazarev, Menlo Park, CA (US)

(73) Assignee: Crysoptix KK

(21) Appl. No.: 12/921,091

(22) PCT Filed: Mar. 4, 2009
(86) PCT No.: PCT/GB09/50219

§ 371 Date: Nov. 29, 2010

(30) Foreign Application Priority Data
Mar. 4, 2008 (GB) 0804083.4

(51) Int. Cl. C09K 19/32; C07D 471/06; C07D 487/22; C07D 241/38; C07D 487/04; C07D 251/54; C07D 403/12; C07D 251/24; C07C 43/20; C07C 63/48; C07C 43/205; C09K 19/00; C09K 19/34; C09K 19/12

(52) U.S. Cl. 428/1.1; 546/28; 544/198; 544/342; 544/197; 544/216; 568/633; 562/488; 568/643; 252/299.01; 252/299.61; 252/299.66; 252/299.62

(57) ABSTRACT

This invention relates to polycyclic organic compounds of general structural formula (I): wherein Y is a predominantly planar polycyclic system being at least partially aromatic, W₁, W₂, and W₃ are different groups providing solubility in an organic solvent, and sum (n1+n2+n3) is 1, 2, 3, 4, 5, 6, 7 or 8. The polycyclic organic compounds are substantially transparent for electromagnetic radiation in the visible spectral range and are capable of forming supramolecules in the organic solvent.

The present invention relates to organic chemistry, in particular, to polycyclic organic compounds, solution thereof and compensation panel comprising retardation layers based on these compounds. More specifically, the present invention is related to the optical compensators for liquid crystal displays.

Optical compensators are used to alter the relative phase of polarized light passing through said compensators, and thus, are well suited for use in applications where control over the polarization is required. For example, optical compensators comprising at least one retardation layer are used to introduce a phase delay in incident light to correct the phase differences between two components of polarized light introduced by other optical components in a system.

One particularly important application of optical retardation layers is providing polarization compensation for liquid crystal display (LCD) panels.

LCD panels are widely used in watches and clocks, photographic cameras, technical instruments, computers, flat TV, projection screens, control panels and large area of information-providing devices. The information in many LCD panels is presented in the form of a row of numerals or characters, which are generated by a number of segmented electrodes arranged in a pattern. The segments are connected by individual leads to driving electronics, which applies a voltage to the appropriate combination of segments to display the desired information by controlling the light transmitted through the segments. Graphic information or television displays may be achieved by a matrix of pixels, which are connected by an X—Y sequential addressing scheme between two sets of perpendicular conductors. More advanced addressing schemes use arrays of thin film transistors to control the drive voltage at the individual pixels. This scheme is applied predominantly to twisted nematic liquid crystal displays, but is also finding use in high performance versions of super twist nematic liquid crystal displays.

Ideal display should show equal contrast and colour rendering, while looking on them under different angles deviating from the normal observation direction. The different kinds of displays based on nematic liquid crystal, however, possess an angle dependence of the contrast. This is, at angles deviating from the normal observation direction, the contrast becomes lower and the visibility of the information is diminished. The materials commonly used in nematic LCDs are optically positively uniaxially birefringent, which means that an extraordinary refractive index nₑ is larger then the ordinary refractive index nₒ; Δn=nₑ−nₒ>0. The visibility of the displays under oblique angles can be improved by using optical compensators with negative birefringence (Δn<0). Besides that, the loss of contrast is caused by light leaking through the black state pixel elements at large viewing angles. In colour liquid crystal displays the leakage also causes severe colour shifts for both saturated and grey scale colours. These limitations are particularly important for displays used for the control panels in aircraft applications, where co-pilot viewing of the pilot's displays is important. It would be a significant improvement in the art to provide a liquid crystal display capable of presenting a high quality, high contrast image over a wide field of view.

The chemical compounds used for the compensators should be transparent in the working spectral wavelength range. Most LCD devices are adapted for a human eye and for these devices the working range is a visible spectral range

The water-based retardation films are not always the optimal solution in some applications due to their low stability in highly humid conditions. Thus there is a need to provide new optical compensators with good environment stability and mechanical strength. The present invention provides overcoming the disadvantages mentioned above.

Definitions of various terms used in the description and claims of the present invention are listed below.

The term “partially aromatic” refers to an aromatic conjugated system within a molecule.

The term “optical axis” refers to a direction in which propagating light does not exhibit birefringence.

The term “visible spectral range” refers to a spectral range having the lower boundary approximately equal to 400 nm, and upper boundary approximately equal to 700 nm.

The term “retardation layer” refers to an optical element that divides an incident monochromatic polarized light into components and introduces a relative retardance or phase shift between them.

The term “retardance” of a retardation element refers to the just-mentioned relative retardance of phase shift. “Quarter-wave plate” refers to a retardation element that has a constant retardance equal to 90°. “Half-wave plate” refers to a retardation element that has a constant retardance equal to 180°.

The term “compensation panel” refers to an optic device which includes retardation layer.

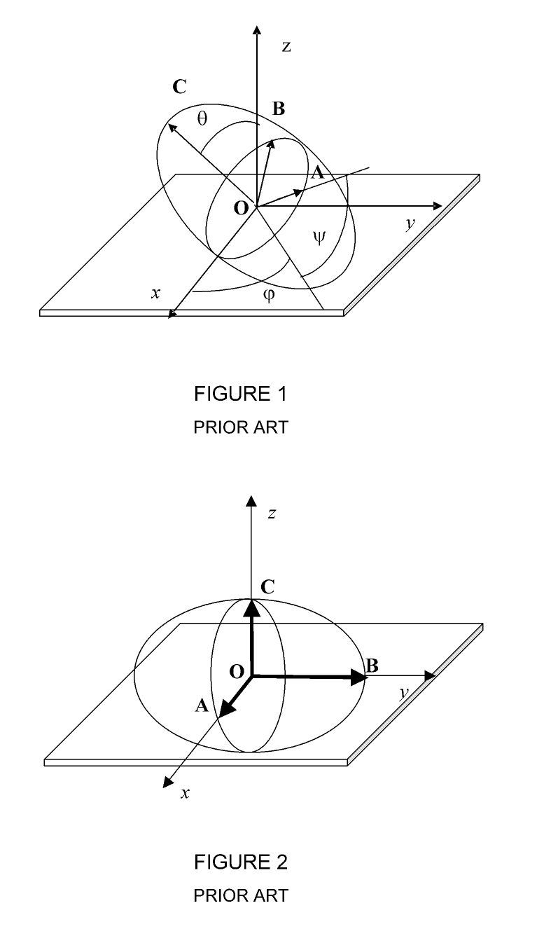

Types of plates in the compensation panel are closely connected to orientations of the principal axes of a particular permittivity tensor with respect to the natural coordinate frame of the plate. The natural xyz coordinate frame of the plate is chosen so that the z-axis is parallel to the normal direction and the xy plane coincides with the plate surface. FIG. 1 (prior art) demonstrates a general case when the principal axes (A, B, C) of the permittivity tensor are arbitrarily oriented relative to the xyz frame.

Orientations of the principal axes can be characterized using three Euler's angles (θ, φ, ψ) which, together with the principal permittivity tensor components (∈_{A}, ∈_{B}, ∈_{C}), uniquely define different types of optical compensators (FIG. 1). The case when all the principal components of the permittivity tensor have different values corresponds to a biaxial compensator, whereby the plate has two optical axes. For example, in case of ∈_{A}<∈_{B}<∈_{C}, these optical axes are in the plane of C and A axes symmetrically on both sides from the C axis. In the uniaxial case with ∈_{A}=∈_{B}, there is a degenerate case when the two axes coincide, and the C axis is a single optical axis.

The zenith angle θ between the C axis and the z axis is important for the definitions of various compensator types. In the case of θ=0 there are several important types of retardation layers, which are most frequently used for compensation of LCD. Hereinafter the x, y and z-axes of the laboratory frame have been chosen coinciding with A, B and C axes respectively.

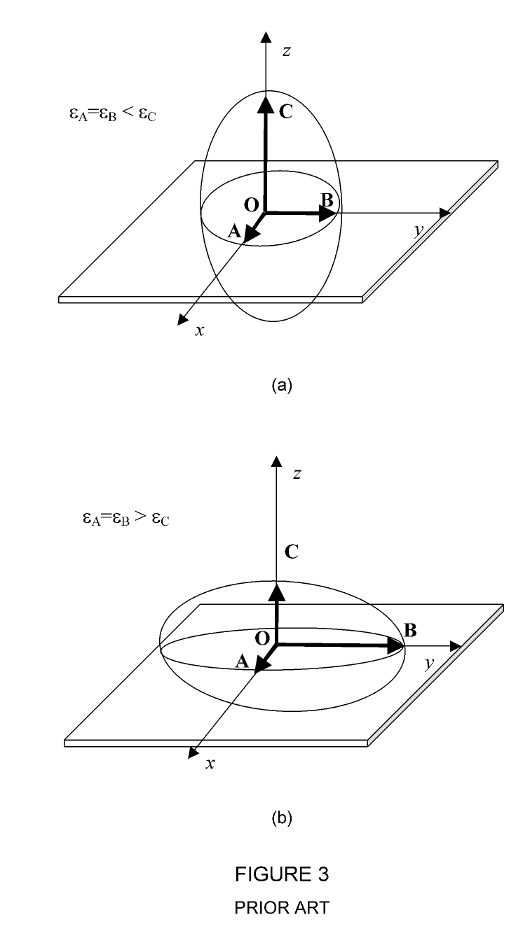

In the case the lowest and highest magnitudes of three principal values ∈_{A}, ∈_{B}, and ∈_{C} of the dielectric permittivity tensor correspond to the A and B axes respectively, then ∈_{A}<∈_{C}<∈_{B}, and two optical axes belong to the AB plane. This retardation layer is named “A_{B}” or “B_{A}” type plate (FIG. 2, prior art). A negative A_{B} plate, when ∈_{A}−∈_{B}<0, is equivalent to a positive B_{A} plate (formally replacing the order of the naming letters changes the sign of the dielectric permittivity difference: ∈_{B}−∈_{A}>0).

There is a different case when two optical axes belong to the plane orthogonal to the plate surface. This case takes place if the lowest or highest magnitude of one of the principal permittivity corresponds to the C-axis. For example, in the case of ∈_{C}<∈_{B}<∈_{A} the retardation layer is named a negative C_{A} or a positive A_{C} plate because two optical axes belong to the plane formed by A and C axes.

There are several important types of uniaxial retardation layers, which are most frequently used for compensation of LCD.

A C-plate is defined by ∈_{A}=∈_{B}≠∈_{C}. In this case, the optical axis coincides with the principal C axis. In the case of ∈_{A}=∈_{B}<∈_{C} the plate is called “positive C-plate”. On the contrary, if ∈_{A}=∈_{B}>∈_{C}, the plate is referred to as a “negative C-plate”. In these two cases the C-axis also corresponds to an extraordinary refractive index. FIG. 3 (prior art) shows an orientation of the principal axes and values of a particular permittivity tensor with respect to the natural coordinate frame of a positive (a) and a negative (b) C-plate.

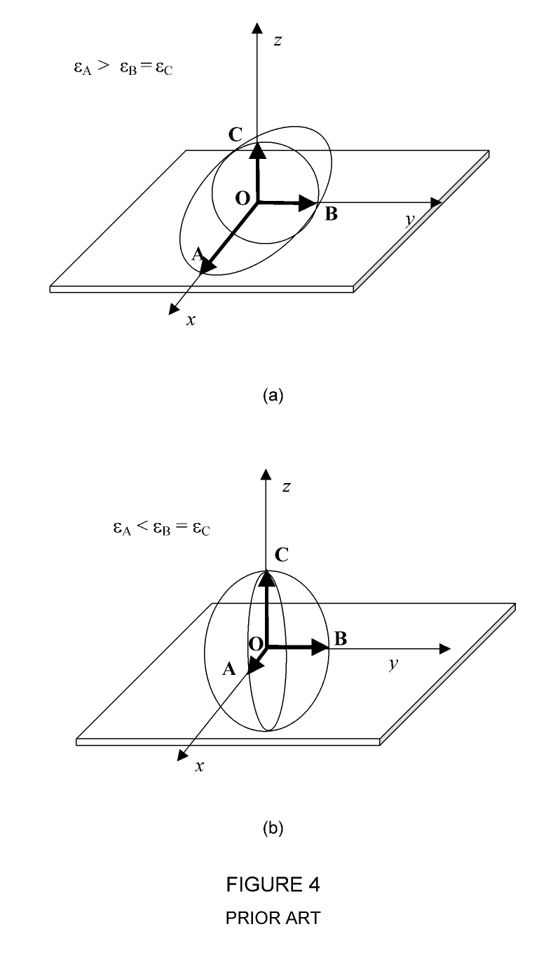

In the case of ∈_{A}≠∈_{B}=∈_{C} the A-principal axis is the optical axis and the plate is named “A-plate”. In the case of ∈_{A}>∈_{B}=∈_{C} the plate is named “positive A-plate” (FIG. 4a, prior art). And in the case of ∈_{A}<∈_{B}=∈_{C} the plate is named “negative A-plate” (FIG. 4b, prior art).

In a general case the permittivity tensor components (∈_{A}, ∈_{B}, and ∈_{C}) are complex values. For uniaxial media the principal permittivity tensor components (∈_{A}, ∈_{B}, and ∈_{C} the principal refraction indices (n_{A}, n_{B}, and n_{C}), and the principal absorption coefficients (k_{A}, k_{B}, and k_{C}) meet the following relation:

$${ɛ}_{i} = {( {n}_{i} - i \frac{λ}{4 π} {k}_{i} )}^{2} , where i ∈ \{ A , B , C \} .$$

The given relation can also be applied to non-conductive biaxial media. In case of conductive biaxial materials the given relation is not valid if the orientation of principal axes of the conductivity tensor is different of that for the dielectric tensor.

The phase speed of an electromagnetic wave propagating along the normal of the anisotropic plate depends on orientation of the wave polarization vector with respect to the principal axes. If the electric field vector of an electromagnetic wave oscillates along the principal axis of the lowest refractive index, then the phase speed of the wave is highest. The corresponding principal axis can be designated as “fast axis”, and the refractive index as “nf”. In a similar way, the largest refractive index defines the “slow” principal axis, and the corresponding designation for the refractive index is “ns”.

Thus the retardation layer may be characterized by two in-plane refractive indices corresponding to a fast principal axis and a slow principal axis (nf and ns), and by one refractive index (nn) in the normal direction. In the case of a biaxial plate all refractive indices nf, ns and nn have different values. As discussed earlier, B_{A}- and A_{C}-plates are biaxial plates. The refractive indices obey the condition ns>nn>nf for a B_{A}-plate, and the condition ns>nf>nn for a positive A_{C}-plate. A- and C-plates are uniaxial plates. The refractive indices of a negative A-plate obey the condition: nn=ns>nf. A-plate can be characterized by the retardation parameter R_{A}=d·(ns−nf), where d is thickness of the retardation layer. In the case of a C-plate there is no in-plane “fast” or “slow” axis (nf=ns). The refractive indices of a negative A-plate obey the condition: nf=ns>nn. C-plate can be characterized by the retardation parameter R_{C}=d·|ns−nn|=d·|nf−nn|, where d is thickness of the retardation layer.

The subject of the invention is illustrated by the following Figures, of which:

FIGS. 1 to 4 are described hereinabove as illustrations to prior art.

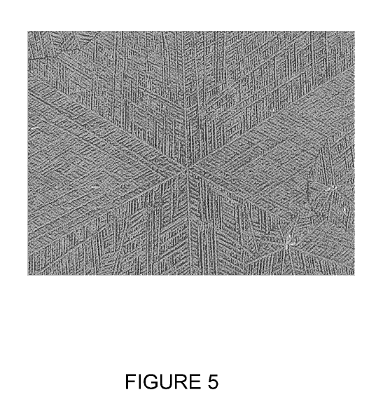

FIG. 5 is a sample of compensation panel with a retardation layer of C-type according to present invention.

A more complete assessment of the present invention and its advantages will be readily achieved as the same becomes better understood by reference to the following detailed description, considered in connection with the accompanying examples and detailed specification, all of which forms a part of the disclosure.

In a first aspect, the present invention provides a polycyclic organic compound of a general structural formula (I)

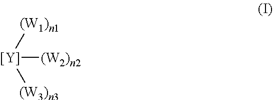

wherein Y is a predominantly planar polycyclic system being at least partially aromatic, W₁, W₂, and W₃ are different groups providing solubility in an organic solvent, and sum (n1+n2+n3) is 1, 2, 3, 4, 5, 6, 7 or 8. The polycyclic organic compound of the present invention is capable of forming supramolecules in the organic solvent, is substantially transparent for electromagnetic radiation in the visible spectral range.

In a second aspect, the present invention provides a solution comprising at least one polycyclic organic compound of a general structural formula (I)

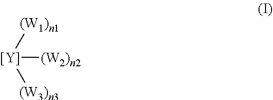

wherein Y is a predominantly planar polycyclic system being at least partially aromatic, W₁, W₂, and W₃ are different groups providing solubility in an organic solvent, and sum (n1+n2+n3) is 1, 2, 3, 4, 5, 6, 7 or 8 Said polycyclic organic compound is capable of forming supramolecules in the organic solvent, and this compound is substantially transparent for electromagnetic radiation in the visible spectral range. The solution of said compound is capable of forming a substantially transparent retardation layer in the visible spectral range.

In a third aspect, the present invention provides a compensation panel comprising at least one retardation layer being substantially transparent in the visible spectral range and comprising at least one polycyclic organic compound of a general structural formula (I):

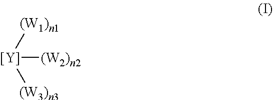

wherein Y is a predominantly planar polycyclic system being at least partially aromatic, W₁, W₂, and W₃ are different groups providing solubility in an organic solvent, and sum n1+n2+n3 is 1, 2, 3, 4, 5, 6, 7 or 8. Said polycyclic organic compound is capable of forming supramolecules in the organic solvent and this compound is substantially transparent for electromagnetic radiation in the visible spectral range.

In one embodiment of the disclosed polycyclic organic compound, the polycyclic system Y is heterocyclic. In another embodiment the heteroatoms in the heterocyclic system are selected from the list comprising N, O and S. In still another embodiment of the disclosed polycyclic organic compound, the polycyclic system Y comprises at least one fragment selected from the list comprising furan, oxirane, 4H-pyran, 2H-chromene, benzo[b]furan, 2H-pyran, thiophene, benzo[b]thiophene, parathiazine, pyrrole, pyrrolidine, pyrazole, imidazole, imidazoline, imidazolidine, pyrazolidine, pyrimidine, pyridine, piperazine, piperidine, pyrazine, indole, purine, benzimidazole, quinoline, phenothiazine, morpholine, thiaziole, thiadiazole, and oxazole.

In yet another embodiment of the disclosed polycyclic organic compound, the polycyclic system Y comprises at least one fragment representing an aromatic hydrocarbon. In another embodiment, the aromatic hydrocarbons are selected from the list comprising acenaphthene, acenaphthylene, acephenanthrylene, biphenylene and naphthalene.

In still another embodiment, the polycyclic system Y comprises fragments selected from the list comprising oligophenyl, imidazole, pyrazole, acenaphthene, triaizine, and having general structural formulas selected from structures 1-24 and shown in the Table 1.

TABLE 1
Examples of polycyclic systems Y with polycyclic aromatic hydrocarbon, imidazole,
pyrazole and triaizine fragments

| | |
|---|---|
| 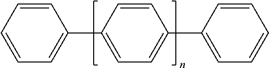 where n is the number in the range from 1 to 8 | 1 |
| 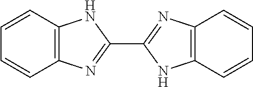 | 2 |
| 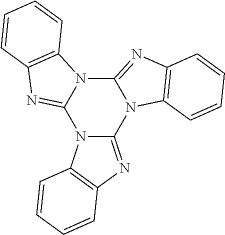 | 3 |
| 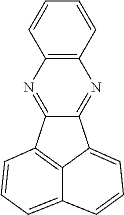 | 4 |
| 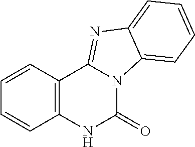 | 5 |
| 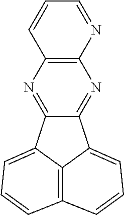 | 6 |
| 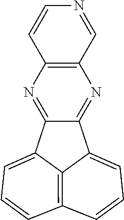 | 7 |
| 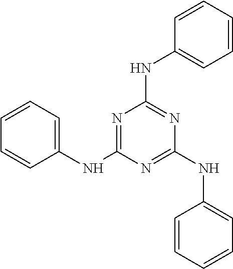 | 8 |
| 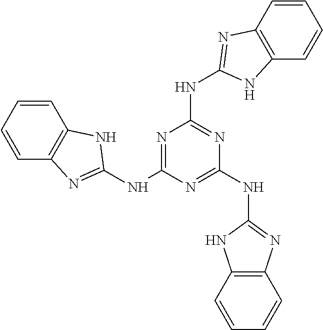 | 9 |
| 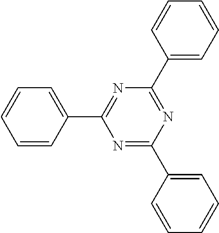 | 10 |
| 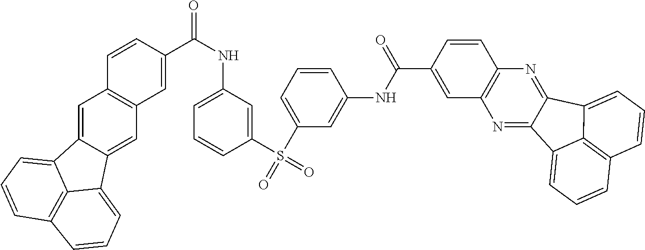 | 11 |
| 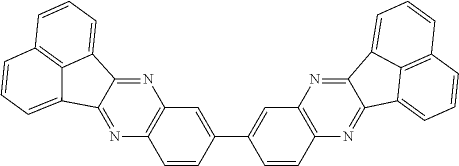 | 12 |
| 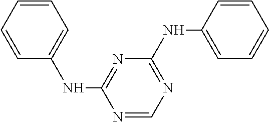 | 13 |
| 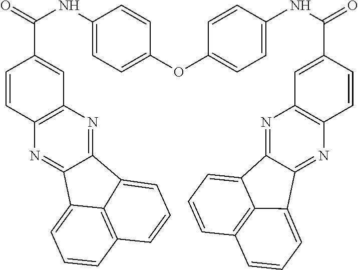 | 14 |
| 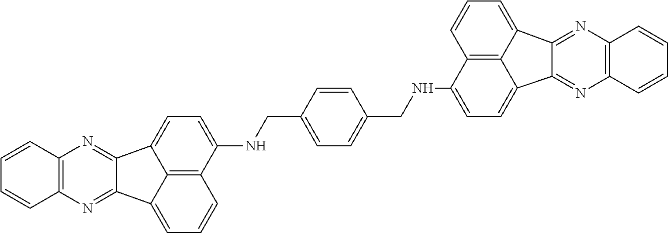 | 15 |
| 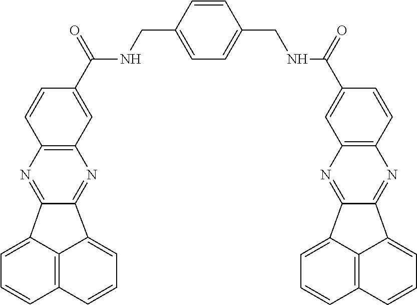 | 16 |
| 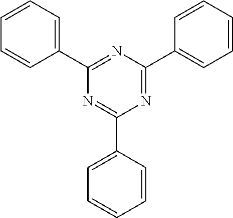 | 17 |
| 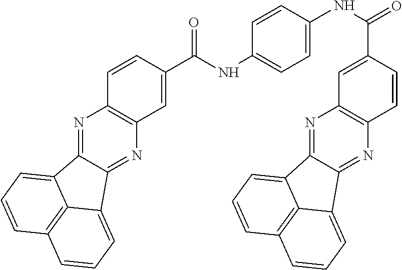 | 18 |
| 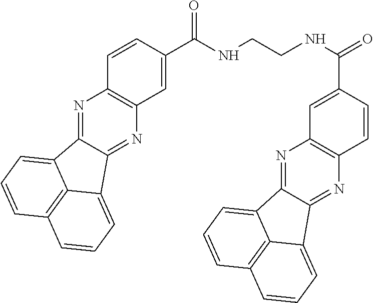 | 19 |
| 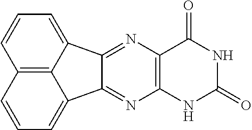 | 20 |
| 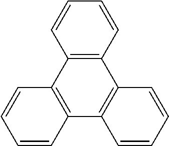 | 21 |
| 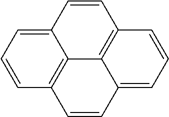 | 22 |
| 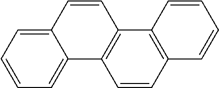 | 23 |
| 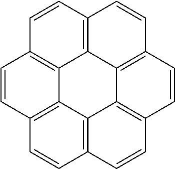 | 24 |

In one embodiment of the disclosed polycyclic organic compound, at least one of the W-groups providing solubility is selected from the list comprising carboxylic (COOH) group, linear and branched (C₁-C₂₀)alkyl, (C₂-C₂₀)alkenyl groups, and (C₂-C₂₀)alkinyl groups. In one embodiment, said W-groups are connected with the polycyclic system Y via at least one covalent bond. In still another embodiment, alkyl groups form a cycle by connecting to the polycyclic system Y via at least two covalent bonds. The hydrophobic interaction between alkyl chains improves solubility by forming supramolecules, and the intermolecular π-π-interactions of unsaturated bonds may play substantial role to ensure the formation of supramolecules in solutions of organic solvents. Hereinafter the term supramolecules comprises molecular aggregations in the solution. The types of supramolecules include rod-like, lamellar supramolecules and the other types known by those skilled in the art.

In another embodiment of the disclosed polycyclic organic compound, at least one of the groups W is connected with the polycyclic system Y via a bridging group A. In yet another embodiment, the bridging group A is selected from the list, comprising —C(O)—, —C(O)O—, —C(O)—NH—, —(SO₂)NH—, —O—, —CH₂O—, —NH—, >N—, and any combination thereof.

In one embodiment of the disclosed polycyclic organic compound, said polycyclic systems may be capable of forming rod-like supramolecules via π-π-interaction. In another embodiment of the disclosed polycyclic organic compound, the rod-like supramolecules have interplanar spacing between the polycyclic systems in the range of approximately 3.1-3.7 A.

The present invention also provides the solution as disclosed hereinabove. In another embodiment of the disclosed solution, the polycyclic system Y is heterocyclic. The heteroatoms in said polycyclic system are selected from the list comprising N, O and S. In still another embodiment of the invention, the polycyclic system Y comprises at least one fragment selected from the list comprising furan, oxirane, 4H-pyran, 2H-chromene, benzo[b]furan, 2H-pyran, thiophene, benzo[b]thiophene, parathiazine, pyrrole, pyrrolidine, pyrazole, imidazole, imidazoline, imidazolidine, pyrazolidine, pyrimidine, pyridine, piperazine, piperidine, pyrazine, indole, purine, benzimidazole, quinoline, phenothiazine, morpholine, thiaziole, thiadiazole, and oxazole.

In another embodiment of the disclosed solution, the polycyclic system Y comprises at least one fragment representing an aromatic hydrocarbon. In still another embodiment of the present invention, the aromatic hydrocarbons are selected from the list comprising acenaphthene, acenaphthylene, acephenanthrylene, biphenylene and naphthalene.

In yet another embodiment of the disclosed solution, the polycyclic system Y is selected from the list comprising oligophenyl, imidazole, pyrazole, acenaphthene, triaizine, and having general structural formula selected from structures 1-24 and shown in the Table 1.

In one embodiment of the disclosed solution, at least one of W-groups providing solubility in the polycyclic organic compound is selected from the list comprising, carboxylic (COOH) group, linear and branched (C₁-C₂₀)alkyl, (C₂-C₂₀)alkenyl, and (C₂-C₂₀)alkinyl. In one embodiment, said W-groups in the disclosed polycyclic organic compound are connected with the polycyclic system Y via at least one covalent bond. In still another embodiment, alkyl groups form a cycle by connecting to the polycyclic system Y via at least two covalent bonds. The hydrophobic interaction between alkyl chains improves solubility by forming supramolecules, and the intermolecular π-π-interactions of unsaturated bonds may play substantial role to ensure the formation of supramolecules in solutions of organic solvents.

In another embodiment of the disclosed solution, at least one of the groups W of the polycyclic organic compounds is connected with the polycyclic system Y via a bridging group A. In yet another embodiment, the bridging group A is selected from the list, comprising —C(O)—, —C(O)O—, —C(O)—NH—, —(SO₂)NH—, —O—, —CH₂O—, —NH—, >N—, and any combination thereof.

In the other embodiment of the disclosed solution, at least one of the groups W is connected with the polycyclic system Y via a bridging group A. In yet another embodiment, the bridging group A is selected from the list, comprising —C(O)—, —C(O)O—, —C(O)—NH—, —(SO₂)NH—, —O—, —CH₂O—, —NH—, >N—, and any combination thereof.

In yet another embodiment of the disclosed solution, the organic solvent is selected from the list comprising ketones, carboxylic acids, hydrocarbons, cyclohydrocarbons, chlorohydrocarbons, alcohols, ethers, esters, and any combination thereof. In still another embodiment of the disclosed solution, the organic solvent is selected from the list comprising acetone, xylene, toluene, ethanol, methylcyclohexane, ethyl acetate, diethyl ether, octane, chloroform, methylenechloride, dichloroethane, trichloroethene, tetrachloroethene, carbon tetrachloride, 1,4-dioxane, tetrahydrofuran, pyridine, triethylamine, nitromethane, acetonitrile, dimethylformamide, dimethulsulfoxide, and any combination thereof.

In one embodiment of the present invention, the solution is a lyotropic liquid crystal solution. In another embodiment of the present invention, the solution is an isotropic solution.

In one embodiment of the disclosed solution, the supramolecules are formed by interaction of at least two said different compounds of formula (I). In another embodiment of disclosed solution, the supramolecules are formed by interaction of the same compounds of the general structural formula (I).

In another embodiment of the invention, the solution further comprises additives, such as surfactants and/or plasticizers which are soluble in the organic solvents. The additives and/or plasticizers are chosen from the compounds which do not damage the alignment of the solution.

The method of forming a retardation layer from the disclosed solution comprises the steps of: a) preparation of a solution of a polycyclic organic compound of the general structural formula (I) in an organic solvent. The polycyclic organic compound is capable of forming supramolecules in the solution, and said compound is substantially transparent in the visible spectral range; b) deposition of a layer of the solution on a substrate; and c) drying with formation of a retardation layer. In one embodiment of the present invention, the method of preparation the disclosed compensation panel further comprises an applying of an external orienting action onto the layer of the solution in order to provide dominant orientation of supramolecules. The orienting action may take place after the step b) of the deposition of the layer of the solution. In another embodiment it may be simultaneously with the step b). The orienting action may be selected from the list comprising external mechanical, electromagnetic, other orienting actions known from the art and any combinations thereof.

The present invention also provides the compensation panel as disclosed hereinabove.

In one embodiment of the compensation panel the polycyclic system Y is heterocyclic. The heteroatoms in said polycyclic system are selected from the list comprising N, O and S. In another embodiment of the compensation panel, the polycyclic system Y comprises at least one fragment selected from the list comprising furan, oxirane, 4H-pyran, 2H-chromene, benzo[b]furan, 2H-pyran, thiophene, benzo[b]thiophene, parathiazine, pyrrole, pyrrolidine, pyrazole, imidazole, imidazoline, imidazolidine, pyrazolidine, pyrimidine, pyridine, piperazine, piperidine, pyrazine, indole, purine, benzimidazole, quinoline, phenothiazine, morpholine, thiaziole, thiadiazole, and oxazole.

In still another embodiment of the disclosed compensation panel the polycyclic system Y comprises at least one fragment representing an aromatic hydrocarbon. In yet another embodiment, the aromatic hydrocarbons are selected from the list comprising acenaphthene, acenaphthylene, acephenanthrylene, biphenylene and naphthalene.

In another embodiment of the disclosed compensation panel the polycyclic system Y comprises fragments selected from the list comprising oligophenyl, imidazole, pyrazole, acenaphthene, triaizine, and having general structural formula selected from structures 1-24 in Table 1.

In one embodiment of the disclosed compensation panel the W-groups providing the solubility in the polycyclic organic compound are selected from the list comprising, carboxylic (COOH) group, linear and branched (C₁-C₂₀)alkyl, (C₂-C₂₀)alkenyl, and (C₂-C₂₀)alkinyl. In another embodiment of the disclosed compensation panel, at least one of the groups W of the polycyclic organic compound is connected with the polycyclic system Y via a bridging group A. In yet another embodiment, the bridging group A is selected from the list, comprising —C(O)—, —C(O)O—, —C(O)—NH—, —(SO₂)NH—, —O—, —CH₂O—, —NH—, >N—, and any combination thereof.

In another embodiment of the invention, the compensation panel comprises two or more retardation layers, wherein at least two of said layers comprise different polycyclic compounds of the general structural formula (I).

In one embodiment of the present invention, the disclosed compensation panel further comprises a substrate. In another embodiment of the disclosed compensation panel, the substrate is transparent for electromagnetic radiation in the visible spectral range. In still another embodiment of disclosed compensation panel the substrate may be made of polymer. In yet another embodiment, the substrate may be made of glass. For the reflective LCDs the substrate may be made of foil having specular or diffuse reflecting surface. In one embodiment, the compensation panel further comprises a transparent adhesive layer applied on top of the retardation layer. In yet another embodiment, the compensation panel further comprises a protective coating applied on the adhesive transparent layer.

In one embodiment of the compensation panel, the retardation layer is at least partially crystalline.

In yet another embodiment of the disclosed compensation panel, the retardation layer is a biaxial retardation layer of BA-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: ns>nn>nf.

In still another embodiment of the disclosed compensation panel, the retardation layer is a biaxial retardation layer of AC-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: ns>nf>nn.

In one embodiment of the present invention, the disclosed compensation panel comprises at least one retardation layer of a first type having slow and fast principal axes lying substantially in the plane of the first type retardation layer, and at least one retardation layer of a second type having an optical axis directed substantially perpendicular to the plane of the second type retardation layer.

In still another embodiment of the disclosed compensation panel, the retardation layer of the first type is a uniaxial retardation layer of negative A-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: nn=ns>nf.

In one embodiment of the disclosed compensation panel, the retardation layer of the first type comprises rod-like supramolecules which are oriented with their longitudinal axes substantially parallel to the fast principal axis. In another embodiment of the present invention, the disclosed compensation panel comprises said rod-like supramolecules having approximately isotropic polarizability in planes which are perpendicular to their longitudinal axes. In still another embodiment of the disclosed compensation panel, the retardation layer of the second type is a uniaxial retardation layer of negative C-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: nf=ns>nn. In yet another embodiment of the disclosed compensation panel, the retardation layer of the second type comprises sheet-like supramolecules with their plane oriented substantially parallel to the surface of said retardation layer.

The following examples are detailed descriptions of methods of preparation and use of certain compounds of the present invention. The examples are presented to illustrate the embodiments of the invention and are not intended as a restriction on the scope of the invention.

It should be understood that the scope of the invention in not limited to these specific structures as many other variations with different W-groups can be readily obtained using the provided procedures.

The following examples describing detailed preparation of the retardation layer and compensation panel are included for the illustration and the person skilled in the art can obtain the retardation layers and compensation panels with any other compound of the present invention.

In the following examples, all the percents are the weight percents and all the temperatures are in the centigrade.

### EXAMPLE 1

Example 1 describes preparation of N,N′-(1-undecyl)dodecyl-5,11-dihexylcoronene-2,3:8,9-tetracarboxydiimide, the predominantly planar polycyclic system of which is presented in Table 1, structural formula 24. The synthetic procedure is shown in Scheme 1 and comprises six steps.

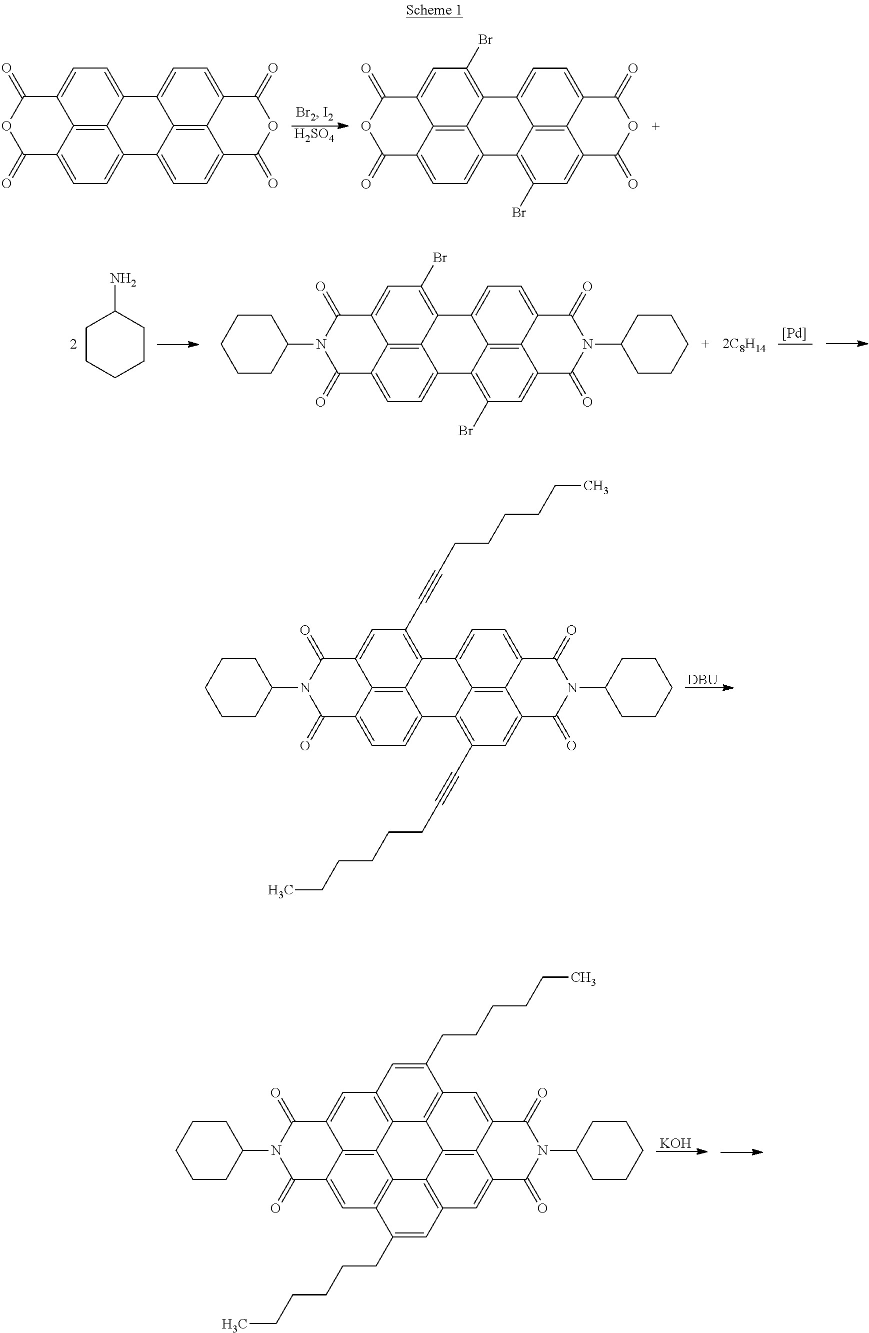

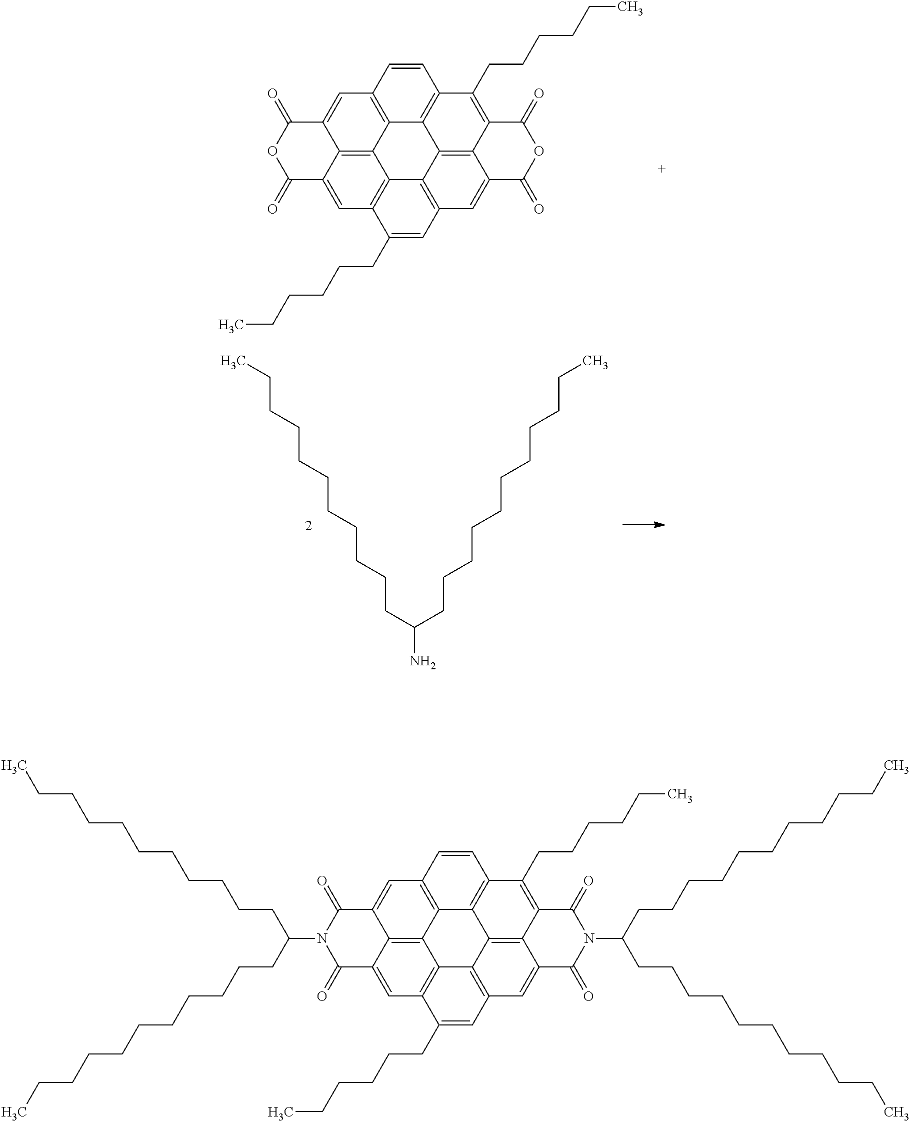

Commercially available Perylene-3,4:9,10-tetracarboxylic dianhydride (100.0 g, 0.255 mol) was brominated with mixture of bromine (29 mL) and Iodine (2.38 g) in 100% sulfuric acid (845 mL) at. ˜85° C. The yield of 1,7-dibromoperylene-3,4:9,10-tetracarboxylic dianhydride was 90 g (64%).

Analysis: calculated: C₂₄H₆Br₂O₆, C, 52.40; H, 1.10; Br, 29.05; O, 17.45%. found: C, 52.29; H, 1.07; Br, 28, 79%. Absorption spectrum (9.82×10⁻⁵ M solution in 93% sulfuric acid): 405 (9572), 516 (27892), 553 (37769).

N,N′-Dicyclohexyl-1,7-dibromoperylene-3,4:9,10-tetracarboxydiimide was synthesized by reaction of 1,7-dibromoperylene-3,4:9,10-tetracarboxylic dianhydride (30.0 g) with cyclohexylamine (18.6 mL) in N-methylpyrrolidone (390 mL) at ˜85° C. The yield of N,N′-dicyclohexyl-1,7-dibromoperylene-3,4:9,10-tetracarboxydiimide was 30 g (77%).

N,N′-Dicyclohexyl-1,7-di(oct-1-ynyl)perylene-3,4:9,10-tetracarboxydiimide by Sonogashira reaction: N,N′-dicyclohexyl-1,7-dibromperylene-3,4:9,10-tetracarboxydiimide (24.7 g) and octyne-1 (15.2 g) in the presence of bis(triphenylphosphine)palladium(II) chloride (2.42 g), triphenylphospine (0.9 g), and copper(I) iodide (0.66 g). The yield of N,N′-dicyclohexyl-1,7-di(oct-1-ynyl)perylene-3,4:9,10-tetracarboxydiimide was 15.7 g (60%).

N,N′-Dicyclohexyl-5,11-dihexylcoronene-2,3:8,9-tetracarboxydiimide was synthesized by heating of N,N′-dicyclohexyl-1,7-di(oct-1-ynyl)perylene-3,4:9,10-tetracarboxydiimide (7.7 g) in toluene (400 mL) in the presence of 1,8-Diazabicyclo[5.4.0]undec-7-ene (0.6 ml) at 100-110° C. for 20 hours.

5,11-dihexylcoronene-2,3:8,9-tetracarboxylic dianhydride was prepared by hydrolysis of N,N′-dicyclohexyl-5,11-dihexylcoronene-2,3:8,9-tetracarboxydiimide (6.4 g, 8.3 mmol) with Potassium hydroxide (7.0 g, 85%) in the mixture of tert-butanol (400 mL) and water (0.4 mL) at 85-90° C. The yield of 5,11-dihexylcoronene-2,3:8,9-tetracarboxylic dianhydride was 4.2 g (83%).

N,N′-(1-undecyl)dodecyl-5,11-dihexylcoronene-2,3:8,9-tetracarboxydiimide by the reaction of 5,11-di(hexyl)coronene-2,3:8,9-tetracarboxylic dianhydride with 12-tricosanamine.

5,11-di(hexyl)coronene-2,3:8,9-tetracarboxylic dianhydride (3.44 g), 12-tricosanamine (7.38 g), benzoic acid (45 mg) and 3-chlorophenol (15 mL) was evacuated and saturated with argon two times at room temperature and 2 times at 100° C. The reaction mixture was agitated at ˜140° C. for 1 hour and 160-165° C. for 20 hours in a flow of argon. After that the reaction mixture was agitated at ˜100° C. and was vacuumed at 10 mm Hg for half an hour. Then apparatus was filled with argon once again and heating was continued for the next 24 hours.

A drop of reaction mixture was mixed with acetic acid (5 mL), centrifuged, solid was dissolved in chloroform (0.5 mL) which was washed with water and dried over sodium sulfate. Thin layer chromatography probe showed good formation of product with Rf 0.9 (eluent: Chloroform-Hexane-Ethylacetate-Methanol (100:50:0.3:0.1 by V)).

The reaction mixture was added in small portions to acetic acid (500 mL) with simultaneous shaking. The orange-red suspension was kept for 3 hours with periodic shaking, then filtered off. The filter cake was washed with water (0.5 L), and then was shaken with water (0.5 L) and chloroform (250 mL) in a separator funnel. The organic layer was separated, washed with water (2×350 mL) and dried over sodium sulfate overnight. The evaporation resulted in 7.0 g of crude product.

Column chromatography was carried out using exactly tuned eluent mixture: chloroform (700 mL), petroleum ether (2 L), ethylacetate (0.6 mL) and methanol (0.2).

Column chromatography was carried out using column: I=20, d=7 cm. Elution of orange fraction and evaporation resulted in orange soft solid material, which was dissolved in chloroform (25 mL) and added slowly to methanol (400 mL) with agitation. The soft precipitate was dried on air overnight, then in vacuum (15 mm Hg) at mild heating)(35°) for 5 hours. The yield of preparation of N,N′-(1-undecyl)dodecyl-5,11-dihexylcoronene-2,3:8,9-tetracarboxydiimide was 5.0 g (70%).

### EXAMPLE 2

Example 2 describes preparation of a compensation panel with a retardation layer of C-type. Coating liquid was prepared as 5% chloroform solution of N,N′-(1-undecyl)dodecyl-5,11-dihexylcoronene-2,3:8,9-tetracarboxydiimide prepared according to Example 1.

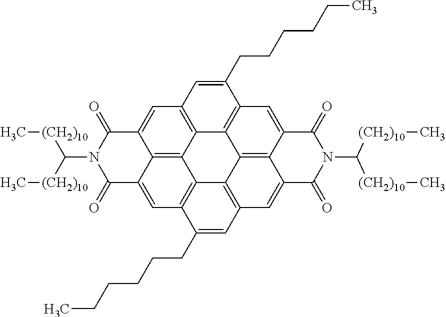

An ITO-coated glass substrates were cleaned following the standard organic-based protocol comprising the steps of soaking in a liquid detergent for 5 minutes, ultrasonic washing with deionized water for 1 hour; drying with compressed air; ultrasonic bath with acetone for 10 min, washing in boiling trichloroethylene during 30 min, ultrasonic bath with acetone for 10 min, washing in boiling isopropanol during 30 min and further drying with compressed air.

A layer of the coating liquid layer was deposited on the fresh treated substrates by Meyer rod technique. The thickness of the resultant retardation layer depends on the coating liquid concentration and Meyer rod gauge. The typical values are from 100 to 1000 nm.

The samples were placed in the furnace and rapidly heated up to 230° C. Then they were cooled down to room temperature at the rate of 5° C./min.

The retardation layers were uniform with defect-free homogeneous area of several sq. cm. as it is shown in FIG. 5.

Polarizing microscopy reveals specific for homeotropic molecular alignment textures. Anisotropy of refractive indices in transparency spectral region is measured to be nf−nn=0.3 (nf=ns).

### EXAMPLE 3

Example 3 describes synthesis of bisbenzimidazo[1′,2′:3,4; 1″,2″:5,6][1,3,5]triazino[1,2-a]benzimidazole-2,8,14-tricarboxylic acid, the predominantly planar polycyclic system of which is presented in Table 1, structural formula 3:

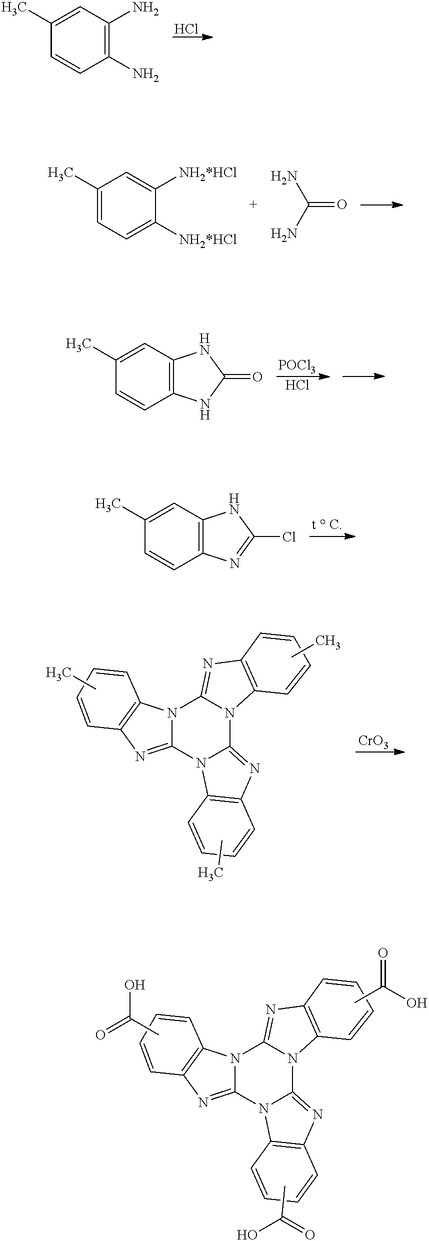

### A. Synthesis of 5-methyl-1,3-dihydro-2H-benzimidazol-2-one

4-Methyl-1,2-phenylendiamine dihydrocloride (20.75 g, 106 mmol) was grinded with urea (7.64 g, 127 mmol). The mixture was charged to a heat-resistant beaker and heated up to 150° C. After 1.5 hours a reaction mixture was cooled to room temperature. The solid material was triturated and charged to heat-resistant beaker and was further heated at 150° C. for 1.5 hours. Then reaction mixture was dissolved in the boiling 1-1.5% aqueous solution of sodium hydroxide (1.5 L). Obtained solution was filtered from an undissolved solid, boiled with activated black carbon (BAU-A, 2 g) for 20-30 min and filtered. Filtrate was acidified by concentrated hydrochloric acid till pH ˜6. White precipitate was filtered, washed with water (100 mL) and dried in desiccator under Phosphorous oxide in vacuo. Yield: 13.1 g (83.5%).

### B. Synthesis of 2-chloro-6-methyl-1H-benzimidazole

5-Methyl-1,3-dihydro-2H-benzimidazol-2-one (13.1 g, 88.5 mmol) and phosphorus oxychloride (130 mL, freshly distillated) was charged into three-neck round-bottom flask. The mixture was heated up to boiling point till homogeneous solution was formed. After that the dried hydrogen chloride was bubbled through inlet gas-pipe into the reaction mixture. The mixture was boiled for 15 hours. Excess of phosphorus oxychloride was distillated in vacuo. Mixture of ice and water (250 mL) was added to residue. The obtained suspension was cooled to room temperature and filtered. Filtrate was alkalinized by aqueous ammonia solution till pH 8, cooled by cold water and filtered crude 2-chloro-6-methyl-1H-benzimidazole. White powder was crystallized from aqueous methanol (water-methanol: 1:1, 200 mL), washed by aqueous methanol and dried in desiccator under Phosphorous oxide in vacuo. Yield: 8.17 g (55%).

### C. Synthesis of 2,8,14-Trimethyl-bisbenzimidazo[1′,2′:3,4; 1″,2″:5,6][1,3,5]triazino[1,2-a]benzimidazole

2-Chloro-6-methyl-1H-benzimidazole (2.7 g, 16.2 mmol) was charged into round-bottom flask and heated up to 200-205° C. for about 1 hour. Reaction mixture was cooled to room temperature. Solid material (2.2 g) was dissolved in the boiling dioxane (70 mL), resulted solution was cooled to room temperature. Solution was filtered, filter was washed by dioxane (25 mL) and washing dioxane was combined with main solution. Water (40 mL) was added dropwise to obtained solution. Precipitate was filtered, washed with acetone and dried in vacuo under Phosphorous oxide at about 70° C. Yield: 1.16 g (54%).

### D. Synthesis of Bisbenzimidazo[1′,2′:3,4:1″,2″:5,6][1,3,5]triazino[1,2-a]benzimidazole-2,8,14-tricarboxylic acid

2,8,14-Trimethyl-bisbenzimidazo[1′,2′:3,4; 1″,2″:5,6][1,3,5]triazino[1,2-a]benzimidazole (1.03 g, 2.6 mmol) was added to mixture (20 mL) of concentrated sulfuric acid and glacial acid (ratio 8:12). Then powder of chromium trioxide (3.5 g) was added slowly with cooling of reaction mixture. The mixture was stirred for 3 hours at room temperature. Water (20 mL) was added dropwise to the reaction mixture with cooling (20-40° C.). Precipitate was filtered and washed with big volume of water and diluted hydrogen chloride solution (30 mL) additionally. Then precipitate was dried in vacuo under Phosphorous oxide. Yield: 0.72 g (57.6%).

### EXAMPLE 4

Example 4 describes preparation of a solid optical retardation layer of negative C-type with bisbenzimidazo[1′,2′:3,4; 1″,2″:5,6][1,3,5]triazino[1,2-a]benzimidazole-2,8,14-tricarboxylic acid prepared as described in Example 3.

1 g of bisbenzimidazo[1′,2′:3,4; 1″,2″:5,6][1,3,5]triazino[1,2-a]benzimidazole-2,8,14-tricarboxylic acid was dissolved in 9 g of dimethylsulfoxide. The suspension was mixed with a magnet stirrer till complete dissolution.

The coatings were produced and optically characterized, as was described in Example 2. The obtained solid optical retardation layer is characterized by thickness equal to approximately 300 nm and the principle refractive indices which obey the following condition: n_{z}<n_{y}≈nₓ. Out-of-plane birefringence equals to 0.15.

### EXAMPLE 5

Example 5 describes preparation of 2,5,9-(dodecyn-1-yl)acenaphtho[1,2-b]quinoxaline, the predominantly planar polycyclic system of which is presented in Table 1, structural formula 4. The synthetic procedure is shown in Scheme 2 and comprises two steps.

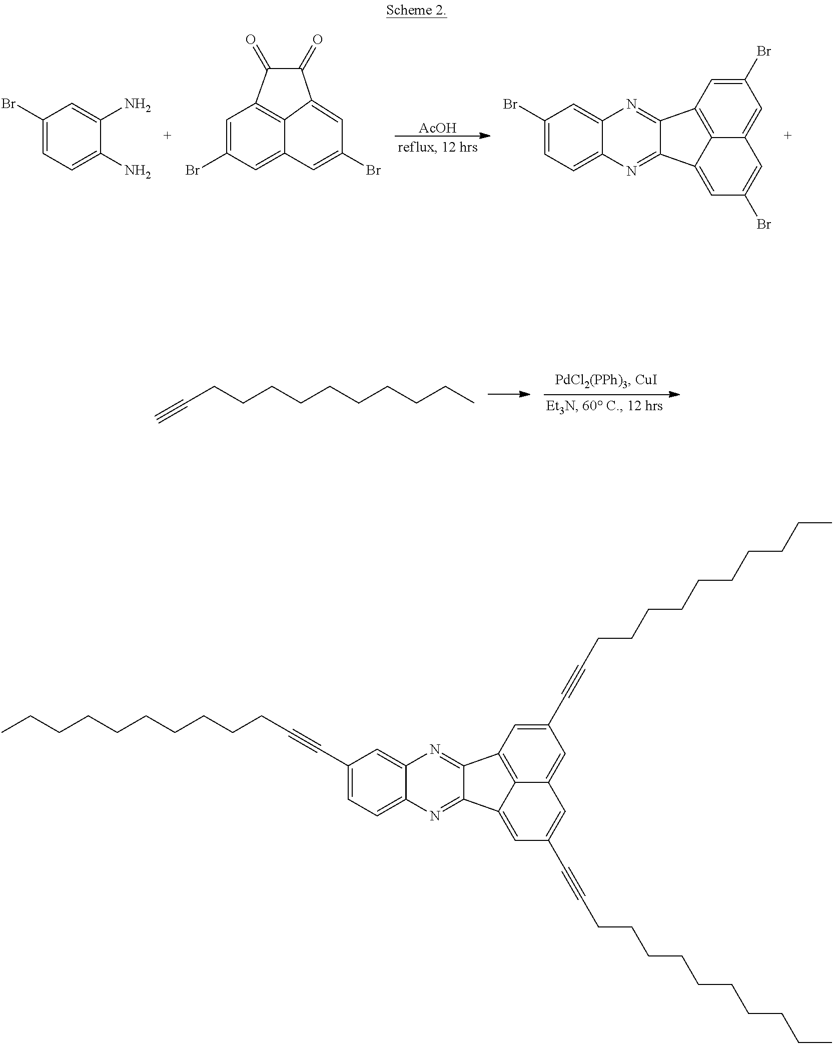

### A. Synthesis of 2,5,9-tribromoacenaphtho[1,2-b]quinoxaline

1-bromo-3,4-diaminobenzene (18.7 g, 100 mmol) was added to a suspension of 4,7-dibromoacenaphthenequinone (34 g) in acetic acid (350 ml). The reaction mixture was refluxed for 12 hours. The solid was separated, washed with acetic acid (80 ml) and dried at 120° C. for 3 hours to yield 36.26 g (74%) of 2,5,9-tribromoacenaphtho[1,2-b]quinoxaline.

### B. Synthesis of 2,5,9-(dodecyn-1-yl)acenaphtho[1,2-b]quinoxaline

Tribromoacenaphtho[1,2-b]quinoxaline (49 g, 100 mmol) was mixed with PdCl₂(PPh₃)₂ (3.7 g, 5 mol %) and CuI (4 g) in 100 ml of dry triethylamine under argon atmosphere. Dodecyne-1 (66 g, 400 mmol) was added and the mixture was stirred at 65° C. overnight. The solvent was removed in vacuo, the residue was dissolved in ethylacetate and washed successively with saturated solutions of NH₄Cl and NaCl. 2,5,9-(dodecyn-1-yl)acenaphtho[1,2-b]quinoxaline was isolated from the concentrated organic phase by column chromatography using hexane-ethylacetate mixture (9:1) as an eluent. Yield: 63.49 g, 85%.

### EXAMPLE 6

Example 6 describes preparation of 2,5,9-tris(decyloxy)acenaphtho[1,2-b]pyrido[2,3-e]pyrazine, the predominantly planar polycyclic system of which is presented in Table 1, structural formula 6. The synthetic procedure is shown in Scheme 3 and comprises two steps.

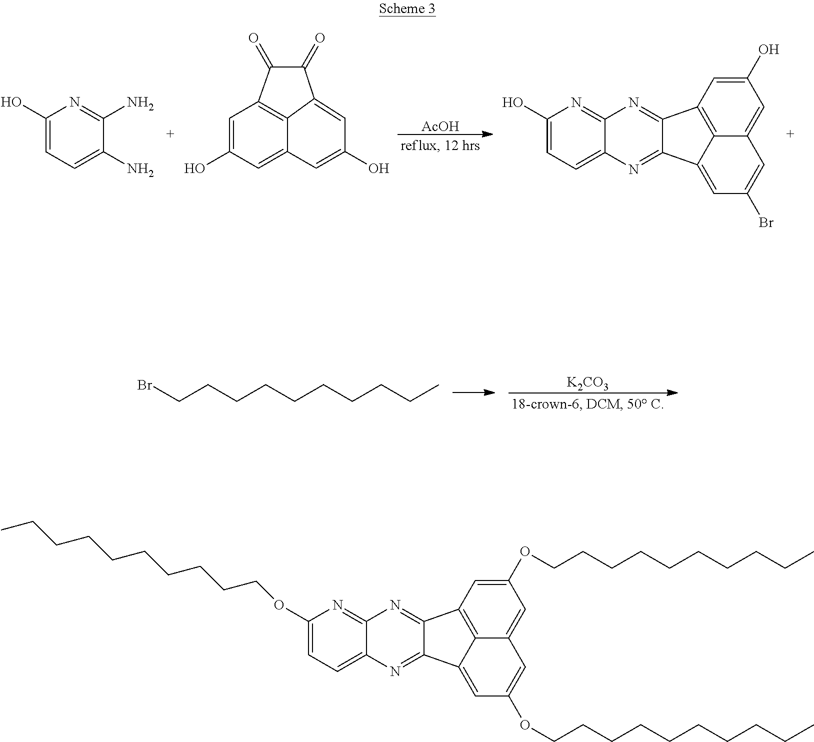

### A. Synthesis of acenaphtho[1,2-b]pyrido[2,3-e]pyrazine-2,5,9-triol

5,6-diaminopyridin-2-ol (12.5 g, 100 mmol) was added to a suspension of 4,7-dihydroxy-acenaphthenequinone (21.4 g, 100 mmol) in acetic acid (250 ml). The reaction mixture was refluxed for 12 hours. The solid was separated, washed with acetic acid (80 ml) and dried at 120° C. for 3 hours to yield 21.23 g (58%) of acenaphtho[1,2-b]pyrido[2,3-e]pyrazine-2,5,9-triol.

### B. Synthesis of 2,5,9-tris(decyloxy)acenaphtho[1,2-b]pyrido[2,3-e]pyrazine

Acenaphtho[1,2-b]pyrido[2,3-e]pyrazine-2,5,9-triol (36.6 g, 100 mmol) was dissolved in DCM (150 ml). 1-bromodecane (66.3 ml, 300 mmol), K₂CO₃ (55.2 g, 400 mmol) and 18-crown-6 (10 mol %, 2.64 g) were added upon stirring. The reaction mixture was stirred at 50° C. for 15 hours. The solvent was removed in vacuo, the residue was dissolved in ethylacetate and washed successively with saturated solutions of NH₄Cl and NaCl. 2,5,9-tris(decyloxy)acenaphtho[1,2-b]pyrido[2,3-e]pyrazine was isolated from the concentrated organic phase by column chromatography using hexane-ethylacetate mixture (9:1) as an eluent. Yield: 57.2 g, 79%.

### EXAMPLE 7

Example 7 describes preparation of acenaphtho[1,2-b]pyrido[4,3-e]pyrazine-2,5,10-tricarboxylic acid, the predominantly planar polycyclic system of which is presented in Table 1, structural formula 7. The synthetic procedure is shown in Scheme 4 and consists of two steps.

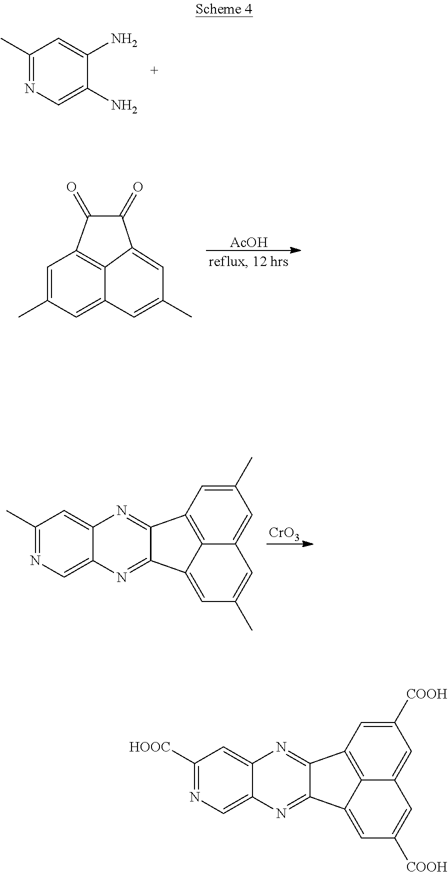

### A. Synthesis of 2,5,10-trimethylacenaphtho[1,2-b]pyrido[4,3-e]pyrazine

6-methylpyridine-3,4-diamine (12.3 g, 100 mmol) was added to a suspension of 4,7-dimethylacenaphthenequinone (21 g, 100 mmol) in acetic acid (150 ml). The reaction mixture was refluxed for 12 hours. The solid was separated, washed with acetic acid (30 ml) and dried at 120° C. for 3 hours to yield 20.2 g (68%) of 2,5,10-trimethylacenaphtho[1,2-b]pyrido[4,3-e]pyrazine.

### B. Synthesis of acenaphtho[1,2-b]pyrido[4,3-e]pyrazine-2,5,10-tricarboxylic acid

2,5,10-trimethylacenaphtho[1,2-b]pyrido[4,3-e]pyrazine (29.7 g, 100 mmol) was added to mixture (200 mL) of concentrated sulfuric acid and glacial acid (ratio 8:12). Then powder of chromium trioxide (50 g) was added slowly with a simultaneous cooling of reaction mixture. The mixture was stirred for 3 hours at room temperature. Water (200 mL) was added dropwise to the reaction mixture with cooling (20-40° C.). Precipitate was filtered, and washed with water and diluted hydrochloric acid (300 mL). The product was dried in vacuo over phosphorous oxide. Yield: 20.12 g (52%).

### EXAMPLE 8

Example 8 describes preparation of N,N,N-tris(3,5-bis(octyloxy)phenyl)-1,3,5-triazine-2,4,6-triamine, the predominantly planar polycyclic system of which is presented in Table 1, structural formula 8. This example is also representative for synthesis of compounds possessing polycyclic aromatic systems with structural formulas 9 and 13, depicted in Table 1. The synthetic procedure is shown in Scheme 5 and consists of two steps.

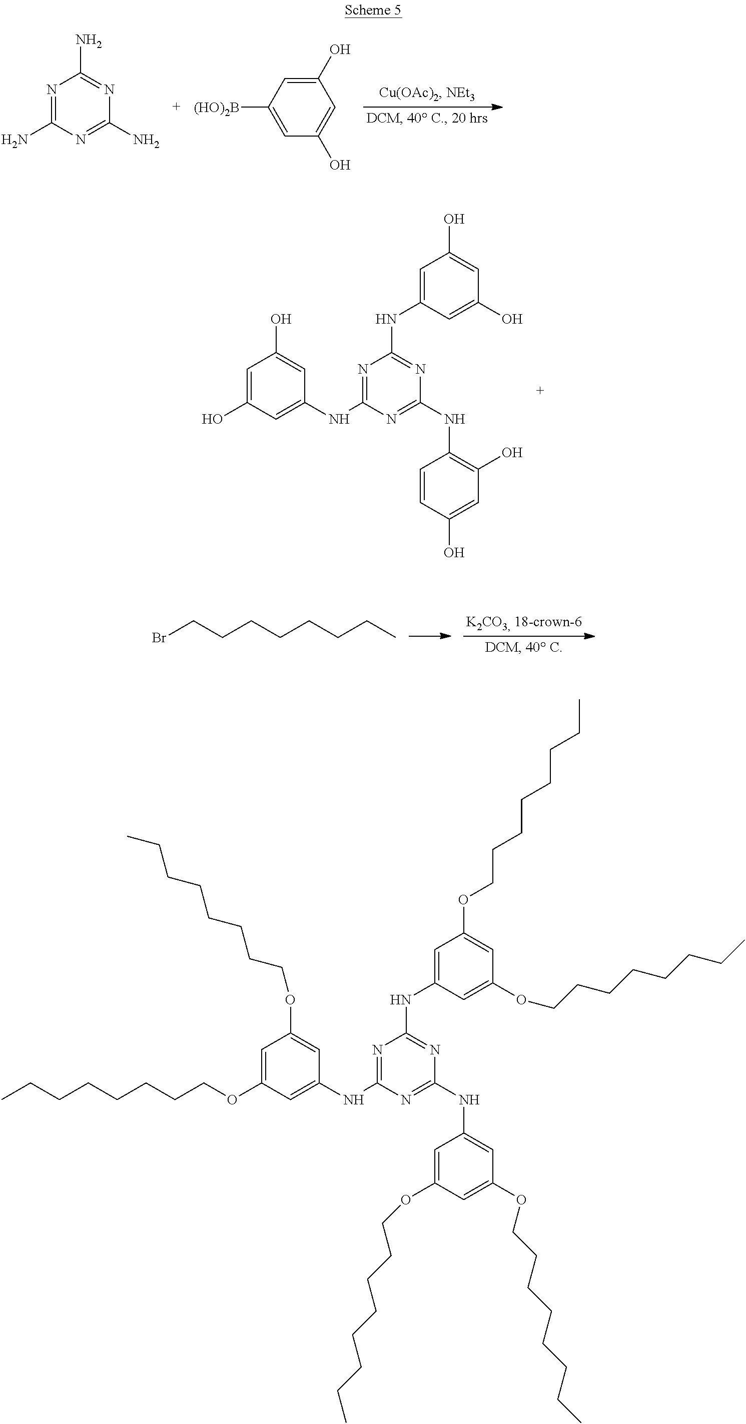

### A. Synthesis of 5,5′,5″-(1,3,5-triazine-2,4,6-triyl)tris(azanediyl)tribenzene-1,3-diol

Commercially available 1,3,5-triazine-2,4,6-triamine (12.6 g, 100 mmol) and (3,5-dihydroxyphenyl)boronic acid (15.3 g, 100 mmol) were dissolved in DCM (100 ml), triethylamine (10 ml) and Cu(OAc)₂ (10 mol %, 1.82 g) were added. The reaction mixture was stirred at 40° C. for 20 hours and then quenched with saturated solution of NH₄Cl. The mixture was extracted with DCM (3×100 ml). 5,5′,5″-(1,3,5-triazine-2,4,6-triyl)tris(azanediyl)tribenzene-1,3-diol was isolated by column chromatography using hexane-ethylacetate mixture as an eluent. Yield: 38.25 g (85%).

### B. Synthesis of N,N,N-tris(3,5-bis(octyloxy)phenyl)-1,3,5-triazine-2,4,6-triamine

5,5′,5″-(1,3,5-triazine-2,4,6-triyl)tris(azanediyl)tribenzene-1,3-diol (45 g) was dissolved in DCM (450 ml). 1-bromooctane (99 g, 600 mmol), K₂CO₃ (96.6 g, 700 mmol) and 18-crown-6 (10 mol %, 2.64 g) were added upon stirring. The reaction mixture was stirred at 50° C. for 15 hours. The solvent was removed in vacuo, the residue was dissolved in ethylacetate and washed successively with saturated solutions of NH₄Cl and NaCl. N,N,N-tris(3,5-bis(octyloxy)phenyl)-1,3,5-triazine-2,4,6-triamine was isolated from the concentrated organic phase by column chromatography using hexane-ethylacetate mixture (9:1) as an eluent. Yield: 102.2 g, 91%.

### EXAMPLE 9

Example 8 describes preparation of 9,9′-(1,4-phenylenebis(azanediyl))bis(oxomethylene)diacenaphtho-[1,2-b]quinoxaline-2,5-dicarboxylic acid, the predominantly planar polycyclic system of which is presented in Table 1, structural formula 18. This example is also representative for synthesis of compounds possessing polycyclic aromatic systems with structural formulas 11, 14, 16, and 19, depicted in Table 1. The synthetic procedure is shown in Scheme 6 and consists of four steps.

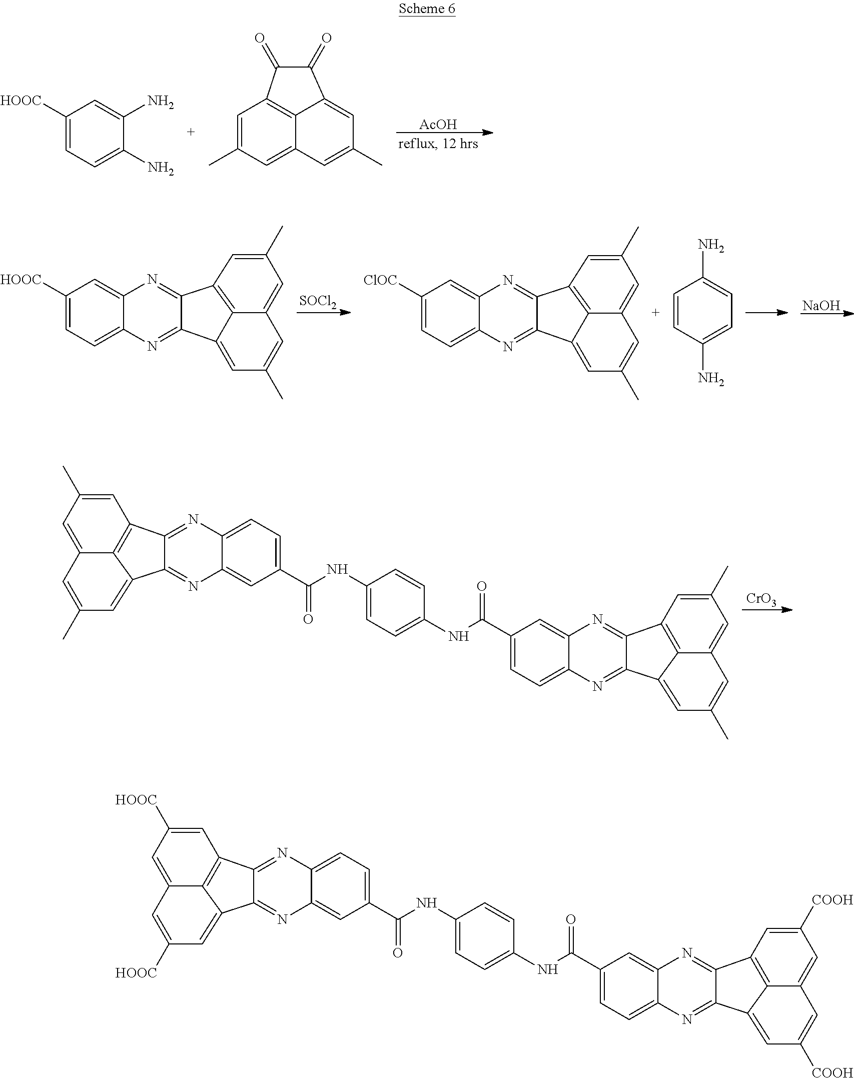

### A. Synthesis of 2,5-dimethylacenaphtho[1,2-b]quinoxaline-9-carboxylic acid

3,4-diaminobenzoic acid (15.2 g, 100 mmol) was added to a suspension of 4,7-dimethylacenaphthenequinone (21 g, 100 mmol) in acetic acid (450 ml). The reaction mixture was refluxed for 12 hours. The solid was separated, washed with acetic acid (130 ml) and dried at 120° C. for 3 hours to yield 15.97 g (49%) of 2,5-dimethylacenaphtho[1,2-b]quinoxaline-9-carboxylic acid.

### B. Synthesis of 2,5-dimethylacenaphtho[1,2-b]quinoxaline-9-carbonyl chloride

2,5-dimethylacenaphtho[1,2-b]quinoxaline-9-carboxylic acid (32.6 g, 100 mmol) was added to thionyl chloride (300 ml) and the mixture was refluxed overnight. The mixture was cooled down to room temperature and filtered. The excessive thionyl chloride was removed in vacuo. 2,5-dimethylacenaphtho[1,2-b]quinoxaline-9-carbonyl chloride were isolated after re-crystallization from hexane Yield: 22.36 g (65%).

### C. Synthesis of N,N′-(1,4-phenylene)bis(2,5-dimethylacenaphtho[1,2-b]quinoxaline-9-carboxamide)

2,5-dimethylacenaphtho[1,2-b]quinoxaline-9-carbonyl chloride (34.4 g, 100 mmol) and 1,4-diaminobenzene (5.4 g, 50 mmol) were dissolved in DCM (100 ml). Upon vigorous stirring an aqueous solution of NaOH (7 g, 25 ml) was added dropwise. The reaction mixture was stirred for 6 hours, the organic layer was separated and washed three times with saturated solution of NH₄Cl and twice with saturated solution of NaCl. Solution was concentrated in vacuo and filtered through silica gel using hexane-ethylacetate mixture as an eluent. The solvents were removed in vacuo, leaving 44 g (61 g) of N,N′-(1,4-phenylene)bis(2,5-dimethylacenaphtho[1,2-b]quinoxaline-9-carboxamide).

### D. Synthesis of 9,9′-(1,4-phenylenebis(azanediyl))bis(oxomethylene)diacenaphtho-[1,2-b]quinoxaline-2,5-dicarboxylic acid

N,N′-(1,4-phenylene)bis(2,5-dimethylacenaphtho[1,2-b]quinoxaline-9-carboxamide) (36.2 g, 50 mmol) was added to mixture (100 mL) of concentrated sulfuric acid and glacial acid (ratio 8:12). Then powder of chromium trioxide (35 g) was added slowly with a simultaneous cooling of reaction mixture. The mixture was stirred for 3 hours at room temperature. Water (200 mL) was added dropwise to the reaction mixture with cooling (20-40° C.). Precipitate was filtered and washed with water and diluted hydrochloric acid (300 mL). 9,9′-(1,4-phenylenebis(azanediyl))bis(oxomethylene)diacenaphtho-[1,2-b]quinoxaline-2,5-dicarboxylic acid was dried in vacuo over phosphorous oxide. Yield: 47.2 (56%).

### EXAMPLE 10

Example 10 describes preparation of 2,4,6-tris(3,5-bis(dodecyloxy)phenyl)-1,3,5-triazine, the predominantly planar polycyclic system of which is presented in Table 1, structural formula 10. The synthetic procedure is shown in Scheme 7 and consists of two steps.

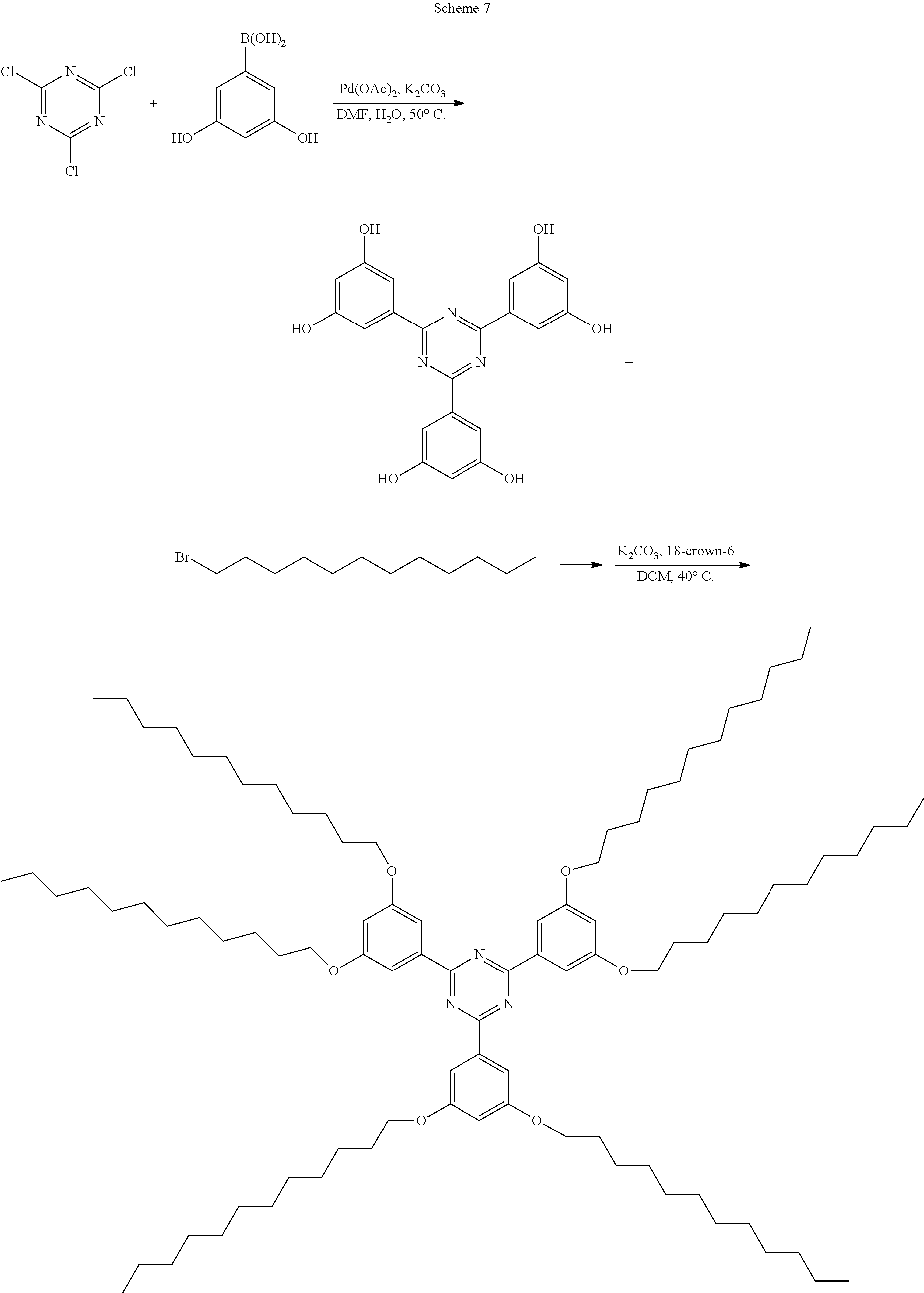

### A. Synthesis of 5,5′,5″-(1,3,5-triazine-2,4,6-triyl)tribenzene-1,3-diol

Commercially available 2,4,6-trichloro-1,3,5-triazine (18.4 g, 100 mmol) and (3,5-dihydroxyphenyl)boronic acid (45.9 g, 300 mmol) were dissolved in DMF (50 ml). Palladium acetate (1.12 g, 5 mol %) and potassium carbonate (55.2 g, 400 mmol) were added and the reaction mixture was stirred at 45° C. overnight. The reaction was extracted with ethylactetate, organic phase was washed successively with saturated solutions of NH₄Cl and NaCl. 5,5′,5″-(1,3,5-triazine-2,4,6-triyl)tribenzene-1,3-diol was isolated by column chromatography using hexane-ethylacetate mixture (9:1) as an eluent. Yield: 35.2 g, 87%.

### B. Synthesis of 2,4,6-tris(3,5-bis(dodecyloxy)phenyl)-1,3,5-triazine

5,5′,5″-(1,3,5-triazine-2,4,6-triyl)tribenzene-1,3-diol (40.5 g, 100 mmol) was dissolved in DCM (350 ml). 1-bromododecane (149.4 g, 600 mmol), K₂CO₃ (96.6 g, 700 mmol) and 18-crown-6 (10 mol %, 2.64 g) were added upon stirring. The reaction mixture was stirred at 50° C. for 15 hours. The solvent was removed in vacuo, the residue was dissolved in ethylacetate and washed successively with saturated solutions of NH₄Cl and NaCl. 2,4,6-tris(3,5-bis(dodecyloxy)phenyl)-1,3,5-triazine was isolated from the concentrated organic phase by column chromatography using hexane-ethylacetate mixture (9:1) as an eluent. Yield: 83.5 g, 59%.

### EXAMPLE 11

Example 11 describes preparation of 4,9-dioctyl-2,7-bis(octyloxy)pyrene, the predominantly planar polycyclic system of which is presented in Table 1, structural formula 22. The synthetic procedure is shown in Scheme 8 and consists of four steps.

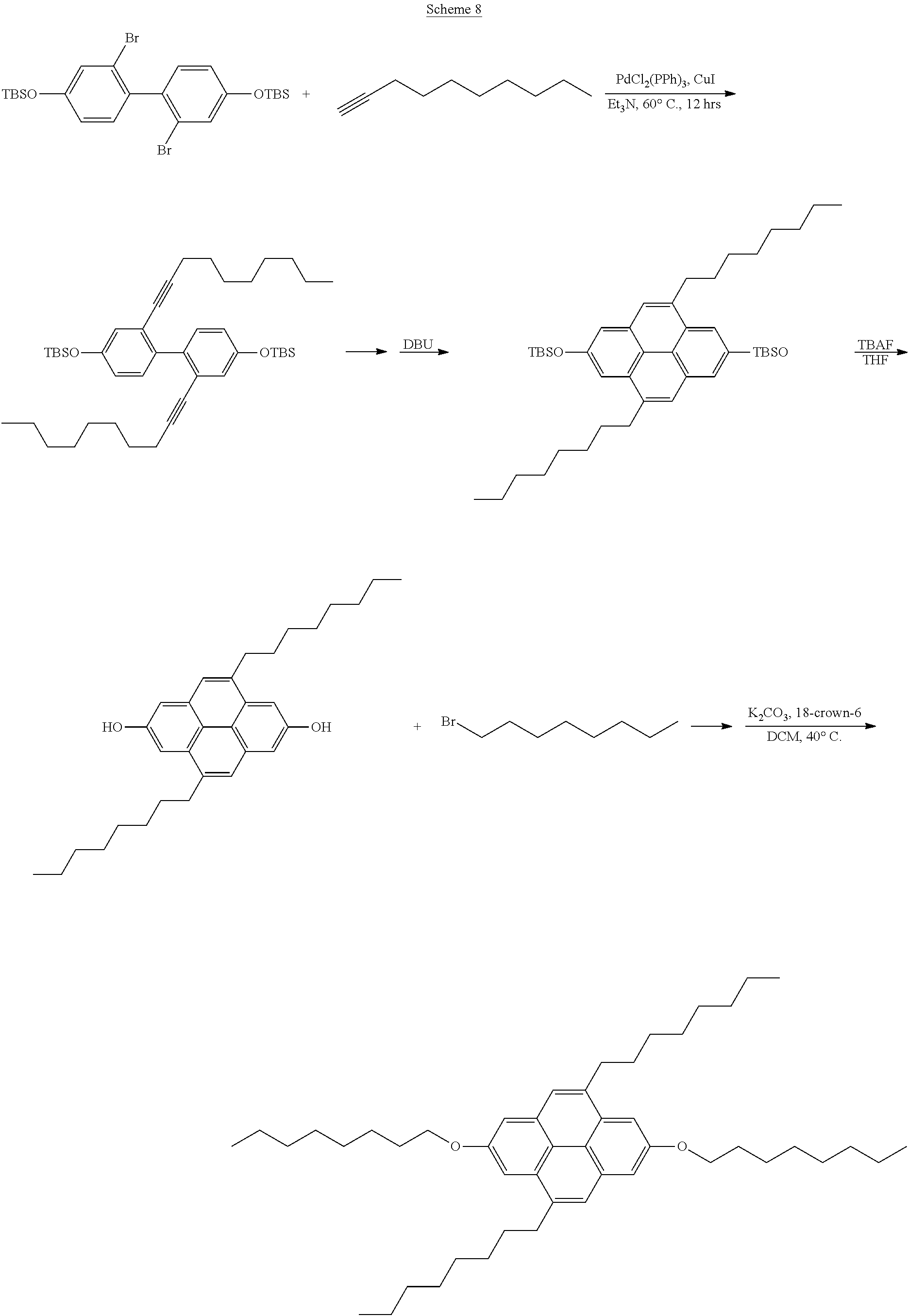

### A. Synthesis of (2,2′-di(dec-1-ynyl)biphenyl-4,4′-diyl)bis(oxy)bis(tert-butyldimethylsilane)

In 100 ml of dry triethylamine under argon atmosphere (2,2′-dibromobiphenyl-4,4′-diyl)bis(oxy)bis(tert-butyldimethylsilane) (43.3 g, 100 mmol) was mixed with PdCl₂(PPh₃)₂ (3.7 g, 5 mol %) and CuI (4 g, 2 mol %). Decyne-1 (27.3 g, 200 mmol) was added and the mixture was stirred at 65° C. overnight. The solvent was removed in vacuo, the residue was dissolved in ethylacetate and washed successively with saturated solutions of NH₄Cl and NaCl. (2,2′-di(dec-1-ynyl)biphenyl-4,4′-diyl)bis(oxy)bis(tert-butyldimethylsilane) was isolated from the concentrated organic phase by column chromatography using hexane-ethylacetate mixture (9:1) as an eluent. Yield: 39.45 g, 72%.

### B. Synthesis of (4,9-dioctylpyrene-2,7-diyl)bis(oxy)bis(tert-butyldimethylsilane)

(4,9-dioctylpyrene-2,7-diyl)bis(oxy)bis(tert-butyldimethylsilane) was synthesized by the heating of (2,2′-di(dec-1-ynyl)biphenyl-4,4′-diyl)bis(oxy)bis(tert-butyldimethylsilane) (27.4 g, 50 mmol) in toluene (700 mL) in the presence of 1,8-Diazabicyclo[5.4.0]undec-7-ene (DBU) (91.2 g, 60 mmol) at 100-110° C. for 20 hours. Yield: 11.50 g (42%).

### C. Synthesis of 4,9-dioctylpyrene-2,7-diol

4,9-dioctylpyrene-2,7-diol was prepared via standard procedure of t-butyldimethylsilyl protection removal with TBAF in THF.

### D. Synthesis of 4,9-dioctyl-2,7-bis(octyloxy)pyrene

4,9-dioctylpyrene-2,7-diol (45.8 g, 100 mmol) was dissolved in DCM (350 ml). 1-bromooctane (38.6 g, 200 mmol), K₂CO₃ (41.4 g, 300 mmol) and 18-crown-6 (10 mol %, 2.64 g) were added upon stirring. The reaction mixture was stirred at 50° C. for 15 hours. The solvent was removed in vacuo, the residue was dissolved in ethylacetate and washed successively with saturated solutions of NH₄Cl and NaCl. 4,9-dioctyl-2,7-bis(octyloxy)pyrene was isolated from the concentrated organic phase by column chromatography using hexane-ethylacetate mixture (9:1) as an eluent. Yield: 49.8 g, 73%.

### EXAMPLE 12

Example 12 describes preparation of chrysene-2,5,8-tricarboxylic acid, the predominantly planar polycyclic system of which is presented in Table 1, structural formula 23. The synthetic procedure is shown in Scheme 9 and consists of three steps.

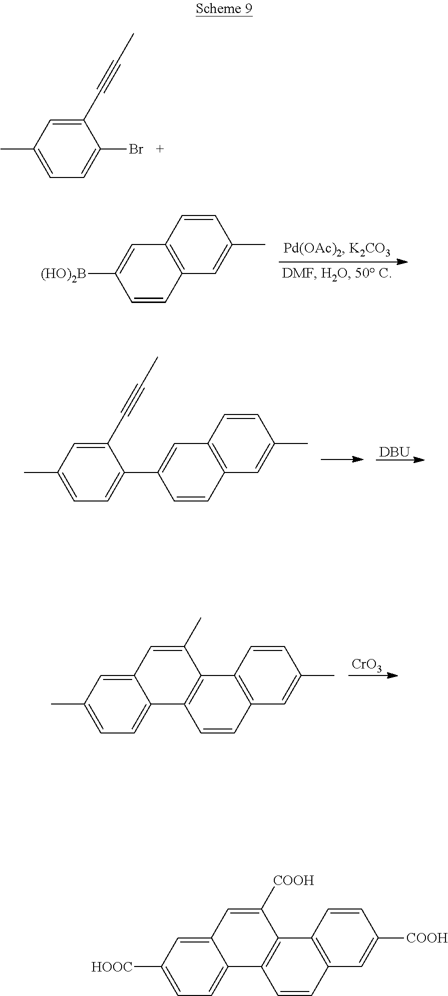

### A. Synthesis of 2-methyl-6-(2-(prop-1-ynyl)phenyl)naphthalene

1-bromo-2-(prop-1-ynyl)benzene (20.9, 100 mmol) and 6-methylnaphthalen-2-ylboronic acid (18.6 g, 100 mmol) were dissolved in DMF (50 ml). Palladium acetate (1.12 g, 5 mol %) and potassium carbonate (27.6 g, 200 mmol) were added and the reaction mixture was stirred at 45° C. overnight. The reaction was extracted with ethylactetate, organic phase was washed successively with saturated solutions of NH₄Cl and NaCl. 2-methyl-6-(2-(prop-1-ynyl)phenyl)naphthalene was isolated by column chromatography using hexane-ethylacetate mixture (20:1) as an eluent. Yield: 18.9 g, 70%.

### B. Synthesis of 2,5,8-trimethylchrysene

2,5,8-trimethylchrysene was synthesized by the heating of 2-methyl-6-(2-(prop-1-ynyl)phenyl)naphthalene (13.5 g, 50 mmol) in toluene (700 mL) in the presence of 1,8-Diazabicyclo[5.4.0]undec-7-ene (DBU) (91.2 g, 60 mmol) at 100-110° C. for 20 hours. Yield: 9.72 g (54%).

### C. Synthesis of chrysene-2,5,8-tricarboxylic acid

2,5,8-trimethylchrysene (27 g, 100 mmol) was added to mixture (200 mL) of concentrated sulfuric acid and glacial acid (ratio 8:12). Then powder of chromium trioxide (35 g) was added slowly with cooling of reaction mixture. The mixture was stirred for 3 hours at room temperature. Water (200 mL) was added dropwise to the reaction mixture with cooling (20-40° C.). Precipitate was filtered and washed with water and diluted hydrochloric acid (300 mL). 2,5,8-tricarboxylic acid was dried in vacuo over phosphorous oxide. Yield: 16.2 g (45%).

### EXAMPLE 13

Example 13 describes preparation of 1,4-di(3,5-dioctyloxyphenyl)benzene, the predominantly planar polycyclic system of which is presented in Table 1, structural formula 1. The synthetic procedure is shown in Scheme 10 and consists of two steps.

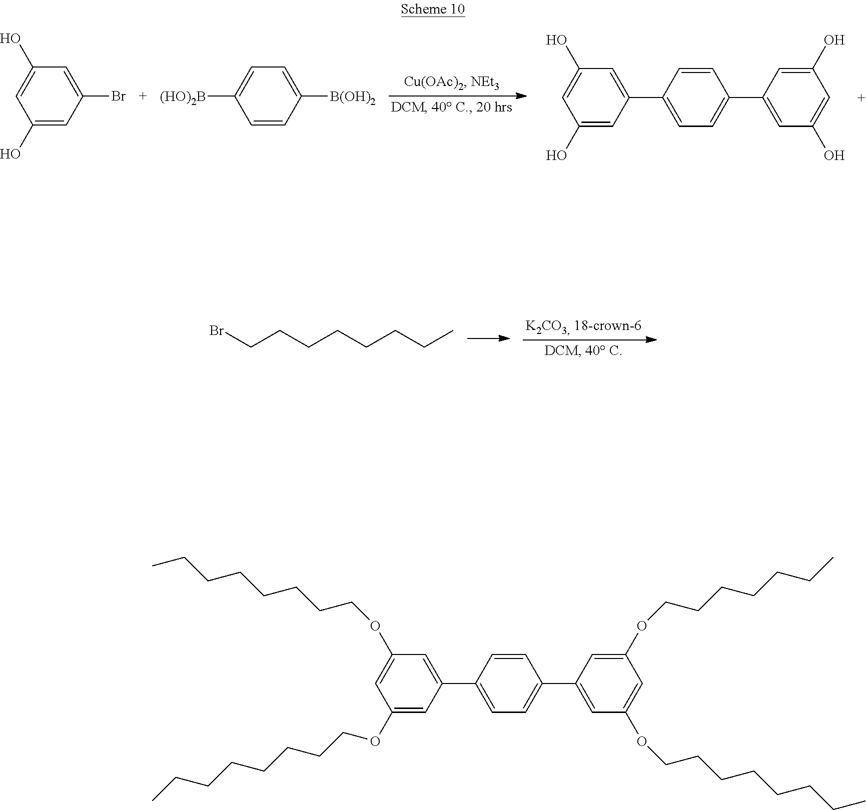

### A. Synthesis of 1,4-di(3,5-dihydroxyphenyl)benzene

5-bromobenzene-1,3-diol (18.9, 100 mmol) and 1,4-phenylenediboronic acid (8.28 g, 50 mmol) were dissolved in DMF (150 ml). Palladium acetate (1.12 g, 5 mol %) and potassium carbonate (27.6 g, 200 mmol) were added and the reaction mixture was stirred at 45° C. overnight. The reaction was extracted with ethylactetate, organic phase was washed successively with saturated solutions of NH₄Cl and NaCl. 2-methyl-6-(2-(prop-1-ynyl)phenyl)naphthalene was isolated by column chromatography using hexane-ethylacetate mixture (7:1) as an eluent. Yield: 11.46 g, 75%.

### B. Synthesis of 1,4-di(3,5-dioctyloxyphenyl)benzene

1,4-di(3,5-dihydroxyphenyl)benzene (29.4 g, 100 mmol) was dissolved in DCM (350 ml). 1-bromooctane (77.2 g, 400 mmol), K₂CO₃ (41.4 g, 500 mmol) and 18-crown-6 (10 mol %, 2.64 g) were added upon stirring. The reaction mixture was stirred at 50° C. for 15 hours. The solvent was removed in vacuo, the residue was dissolved in ethylacetate and washed successively with saturated solutions of NH₄Cl and NaCl. 4,9-dioctyl-2,7-bis(octyloxy)pyrene was isolated from the concentrated organic phase by column chromatography using hexane-ethylacetate mixture (9:1) as an eluent. Yield: 60.1 g, 81%.

While certain preferred embodiments of the invention have been specifically disclosed, it should be understood that the invention is not limited thereto as many variations will be readily apparent to those skilled in the art and the invention is to be given its broadest possible interpretation within the terms of the following claims.

1-57. (canceled)

58. A polycyclic organic compound of the general structural formula I 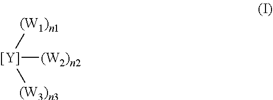
wherein Y is a predominantly planar polycyclic system being at least partially aromatic,
W₁, W₂, and W₃ are different groups providing solubility in an organic solvent, and
sum (n1+n2+n3) is 1, 2, 3, 4, 5, 6, 7 or 8,
wherein said polycyclic organic compound is capable of forming supramolecules in the organic solvent and is substantially transparent for electromagnetic radiation in the visible spectral range.

59. A polycyclic organic compound according to claim 58, wherein the polycyclic system Y is heterocyclic, and wherein one or more heteroatoms of the heterocyclic system are selected from the list comprising N, O and S.

60. A polycyclic organic compound according to claim 58, wherein the polycyclic system Y comprises at least one fragment selected from the list comprising furan, oxirane, 4H-pyran, 2H-chromene, benzo[b]furan, 2H-pyran, thiophene, benzo[b]thiophene, parathiazine, pyrrole, pyrrolidine, pyrazole, imidazole, imidazoline, imidazolidine, pyrazolidine, pyrimidine, pyridine, piperazine, piperidine, pyrazine, indole, purine, benzimidazole, quinoline, phenothiazine, morpholine, thiaziole, thiadiazole, and oxazole.

61. A polycyclic organic compound according to claim 58, wherein the polycyclic system Y comprises at least one fragment representing an aromatic hydrocarbon, and wherein the aromatic hydrocarbon is selected from the list comprising acenaphthene, acenaphthylene, acephenanthrylene, biphenylene and naphthalene.

62. A polycyclic organic compound according to claim 58, wherein the polycyclic system Y comprises fragments selected from the list comprising oligophenyl, imidazole, pyrazole, acenaphthene, triaizine, and having a general structural formula selected from structures 1-24: 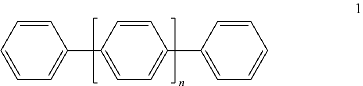
where n is the number in the range from 1 to 8

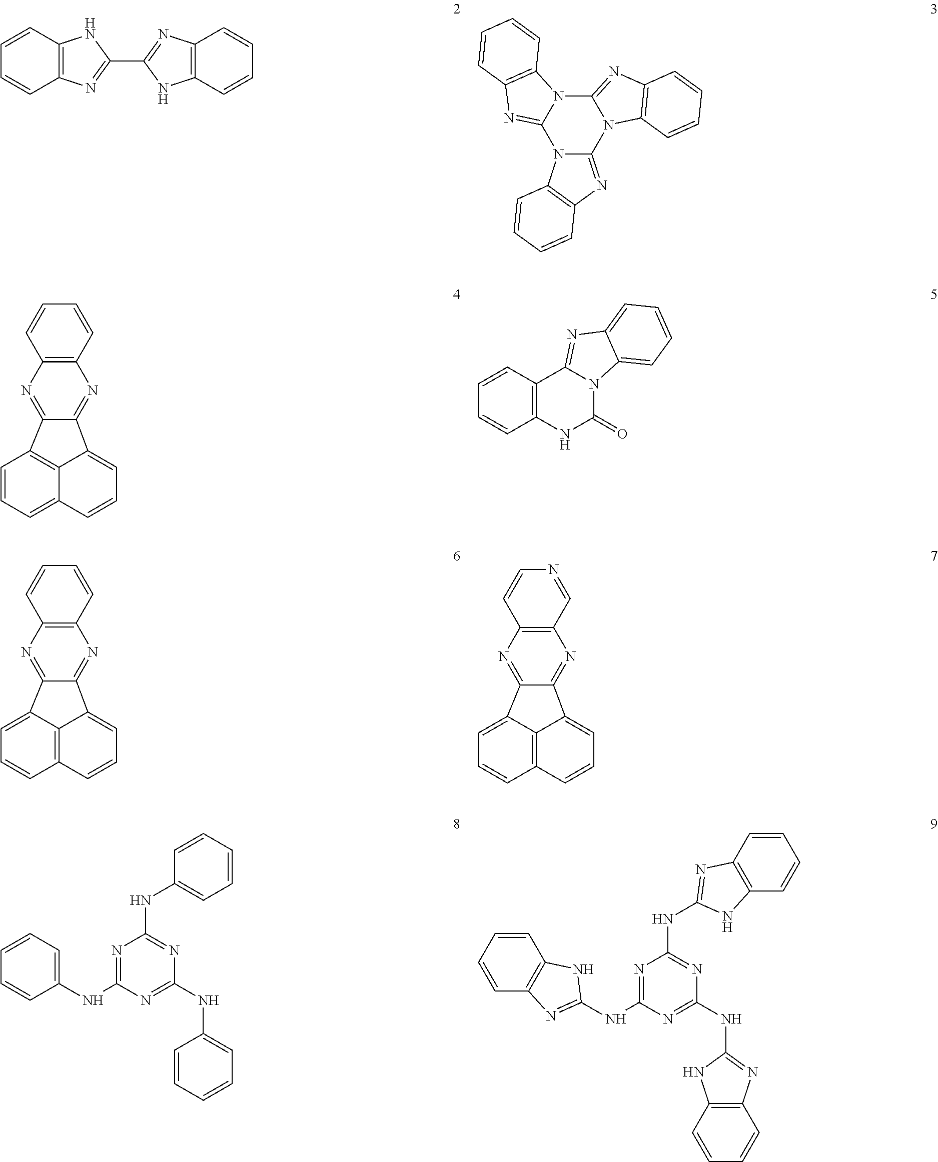

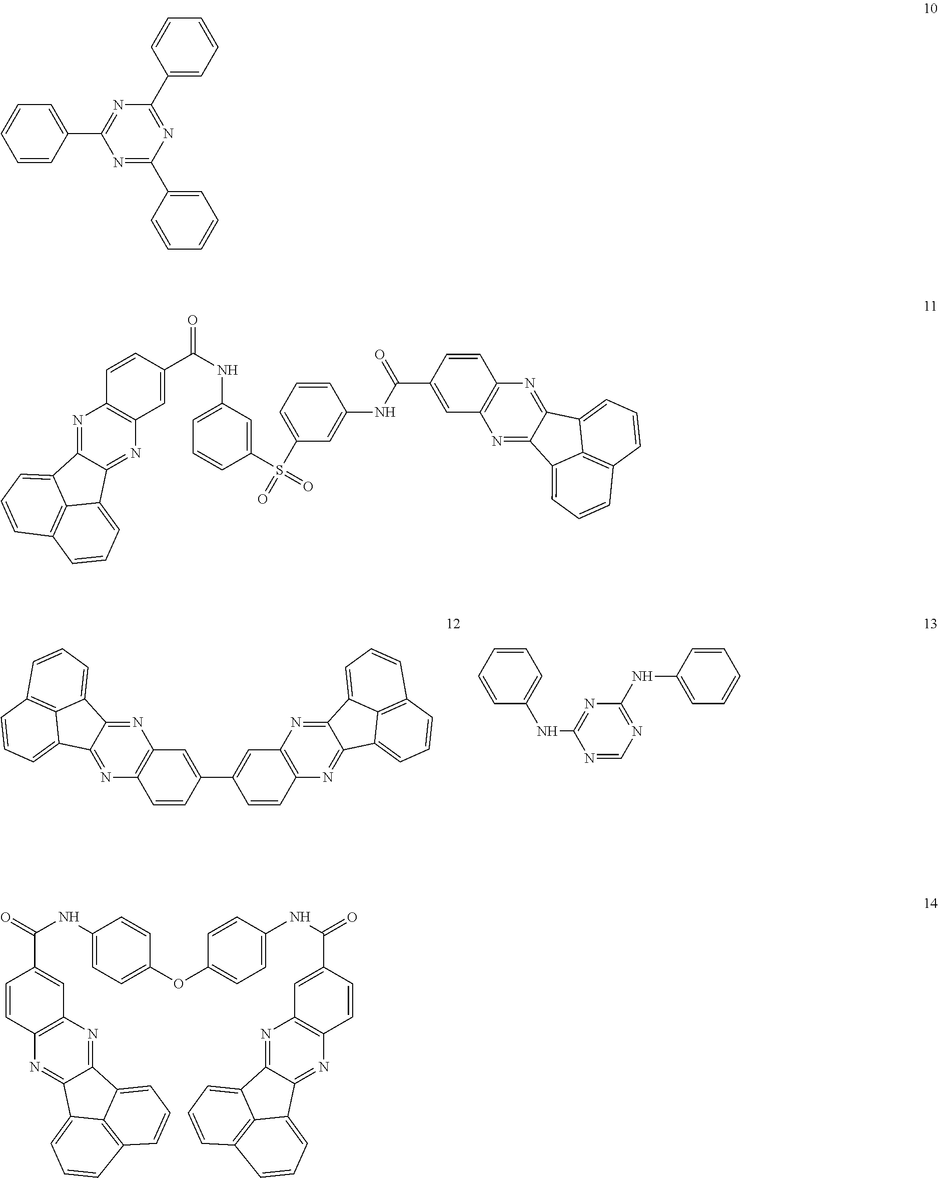

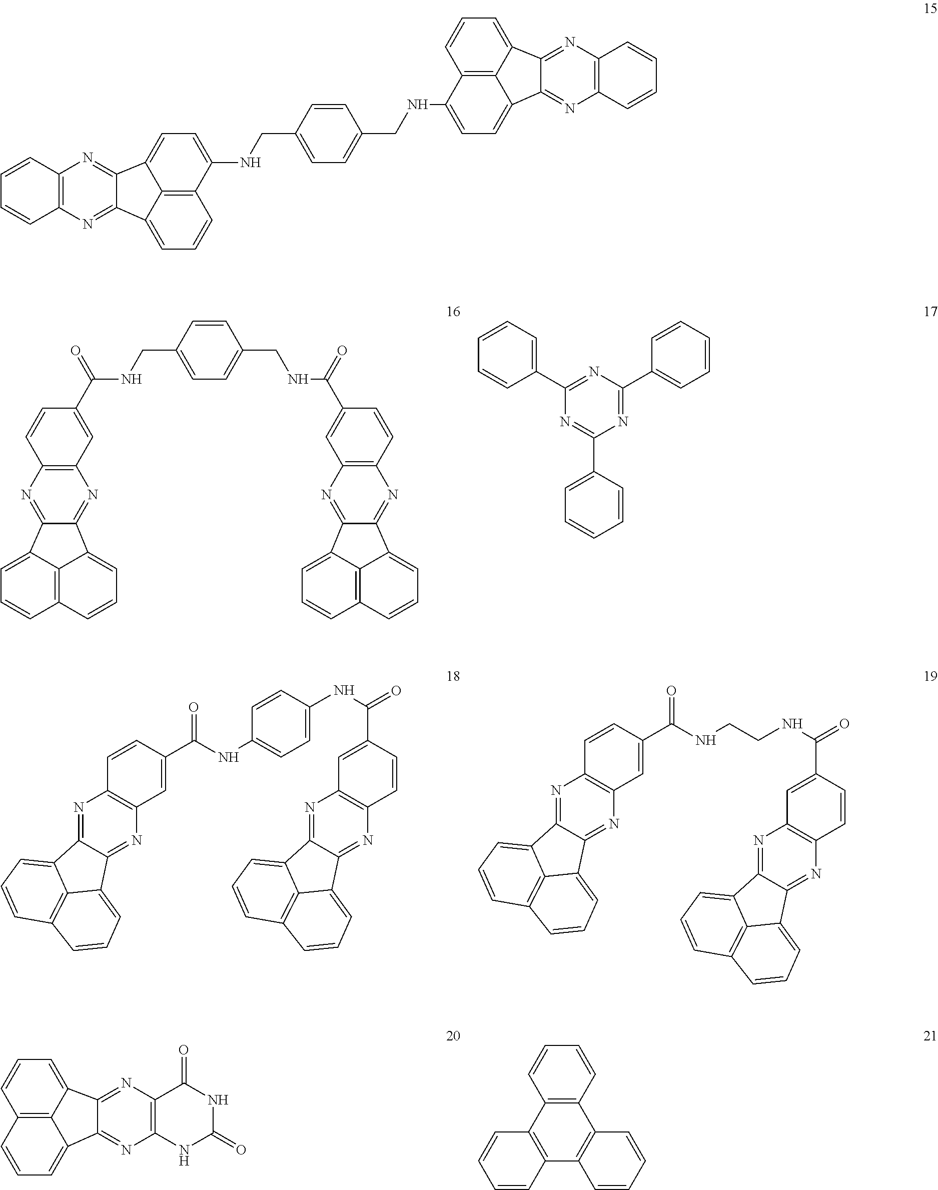

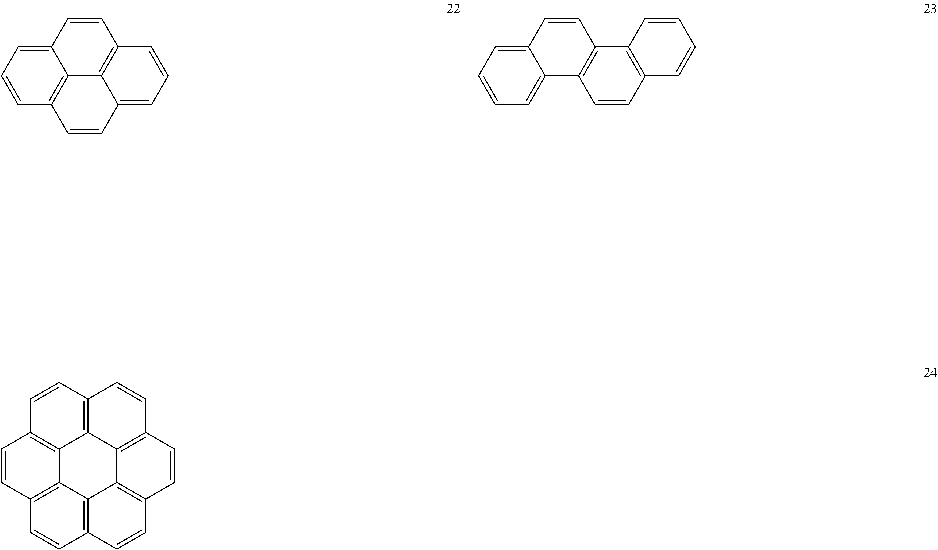

63. A polycyclic organic compound according to claim 58, wherein at least one of the groups W providing solubility is selected from the list comprising carboxylic (COOH) group, linear and branched (C₁-C₂₀)alkyl, (C₂-C₂₀)alkenyl, and (C₂-C₂₀)alkinyl.

64. A polycyclic organic compound according to claim 58, wherein at least one of the groups W providing solubility is connected with the polycyclic system Y via a bridging group A, and wherein the bridging group A is selected from the list comprising —C(O)—, —C(O)O—, —C(O)—NH—, —(SO₂)NH—, —O—, —CH2O—, —NH—, >N—, and any combination thereof.

65. A polycyclic organic compound according to claim 58, wherein the polycyclic system Y is capable of forming rod-like supramolecules via π-π-interaction, and wherein the rod-like supramolecules have interplanar spacing between the polycyclic systems in the range of approximately 3.1-3.7 A.

66. A solution comprising at least one polycyclic organic compound of the general structural formula I 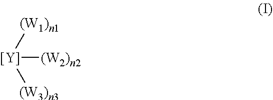
wherein Y is a predominantly planar polycyclic system being at least partially aromatic,
W₁, W₂, and W₃ are different groups providing solubility in an organic solvent, and
sum (n1+n2+n3) is 1, 2, 3, 4, 5, 6, 7 or 8,
wherein said polycyclic organic compound is capable of forming supramolecules in the organic solvent,
said polycyclic organic compound is substantially transparent for electromagnetic radiation in the visible spectral range, and
the solution is capable of forming a substantially transparent retardation layer in the visible spectral range.

67. A solution according to claim 66, wherein the polycyclic system Y is heterocyclic, and wherein heteroatoms of the heterocyclic system Y are selected from the list comprising N, O and S.

68. A solution according to claim 66, wherein the polycyclic system Y comprises at least one fragment selected from the list comprising furan, oxirane, 4H-pyran, 2H-chromene, benzo[b]furan, 2H-pyran, thiophene, benzo[b]thiophene, parathiazine, pyrrole, pyrrolidine, pyrazole, imidazole, imidazoline, imidazolidine, pyrazolidine, pyrimidine, pyridine, piperazine, piperidine, pyrazine, indole, purine, benzimidazole, quinoline, phenothiazine, morpholine, thiaziole, thiadiazole, and oxazole.

69. A solution according to claim 66, wherein the polycyclic system Y comprises at least one fragment representing an aromatic hydrocarbon, and wherein the polycyclic aromatic hydrocarbon is selected from the list comprising acenaphthene, acenaphthylene, acephenanthrylene, biphenylene and naphthalene.

70. A solution according to claim 66, wherein the polycyclic system Y is selected from the list comprising oligophenyl, imidazole, pyrazole, acenaphthene, triaizine, and having general structural formula selected from structures 1-24: 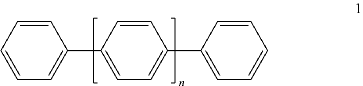
where n is the number in the range from 1 to 8

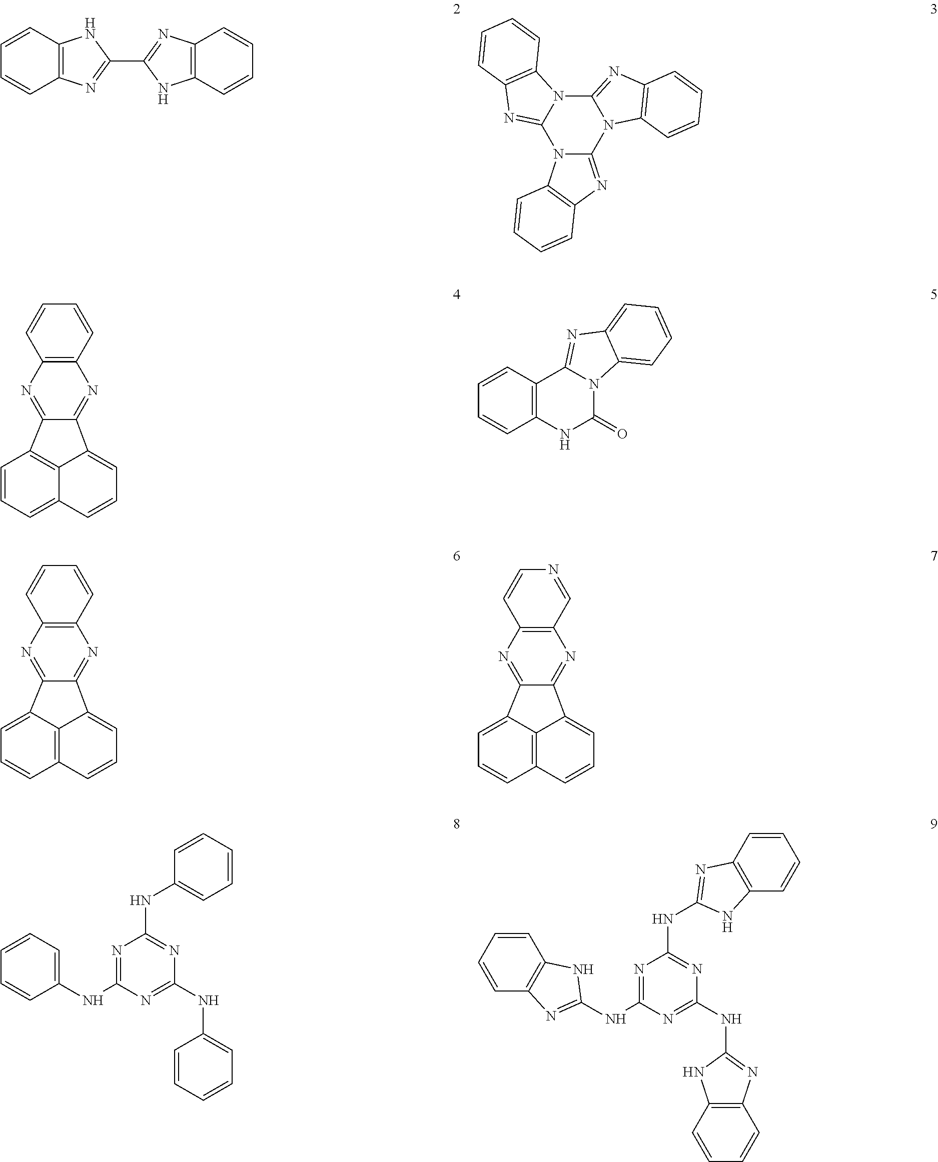

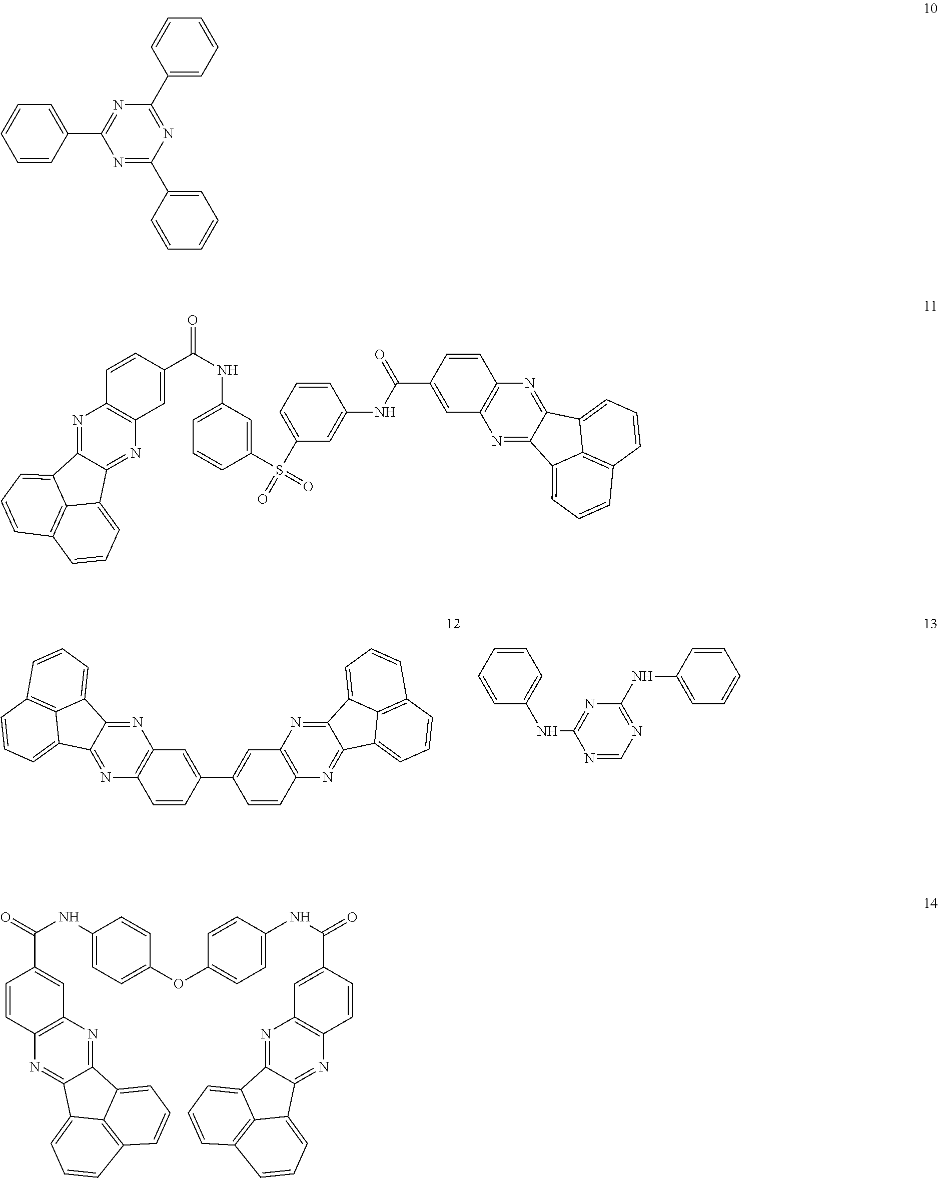

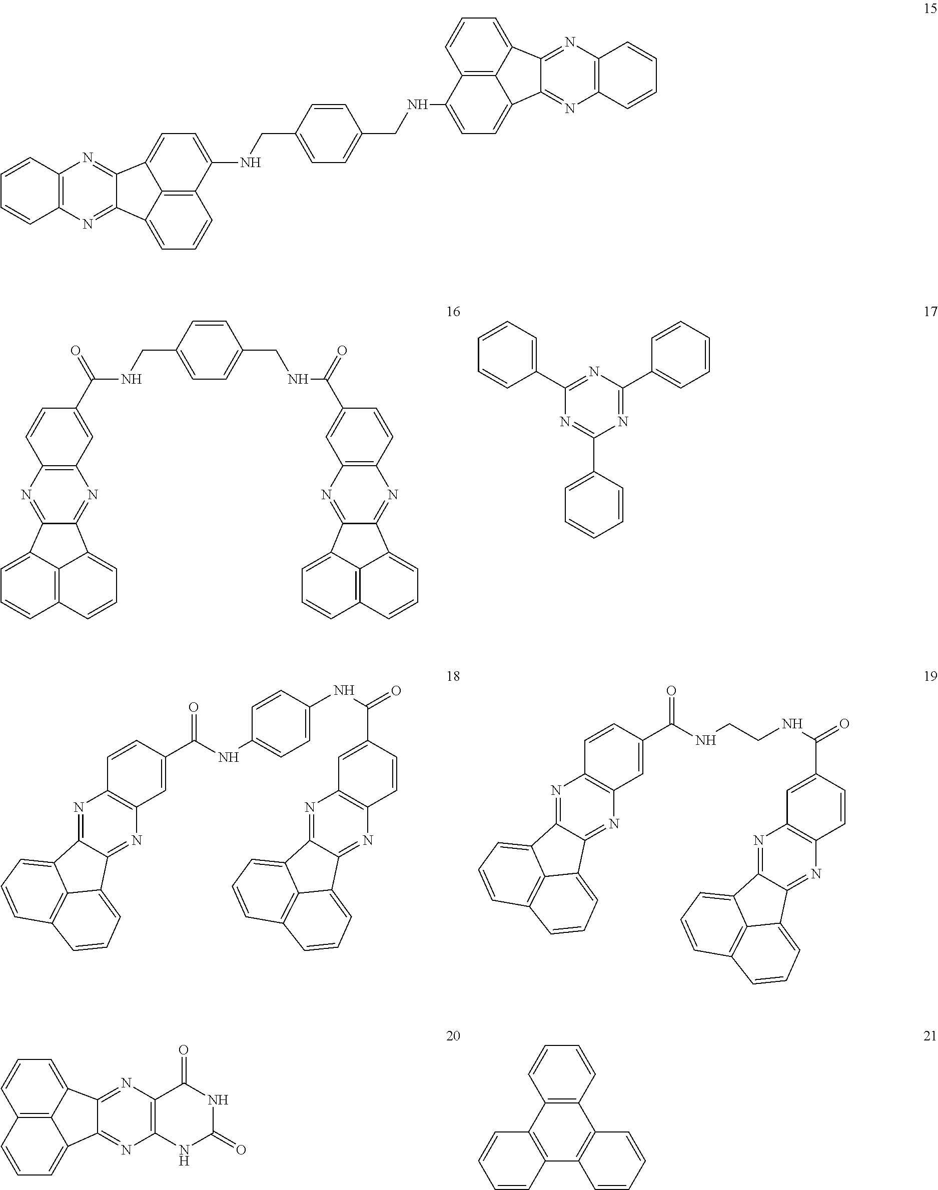

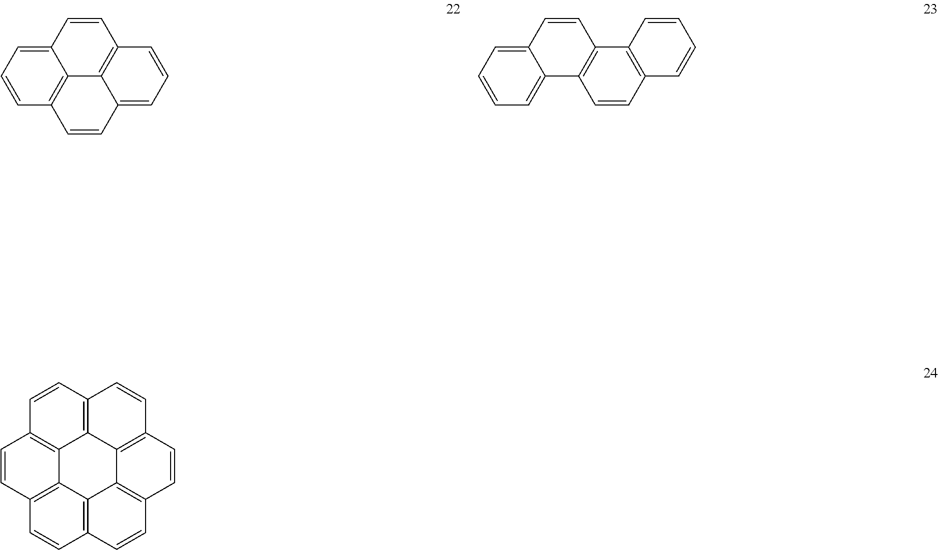

71. A solution according to claim 66, wherein at least one of the groups W providing solubility is selected from the list comprising carboxylic (COOH) group, linear and branched (C₁-C₂₀)alkyl, (C₂-C₂₀)alkenyl, and (C₂-C₂₀)alkinyl.

72. A solution according to claim 66, wherein at least one of the groups W providing solubility in the polycyclic organic compound is connected with the polycyclic system Y via a bridging group A, and wherein the bridging group A is selected from the list comprising —C(O)—, —C(O)O—, —C(O)—NH—, —(SO₂)NH—, —O—, —CH₂O—, —NH—, >N—, and any combination thereof.

73. A solution according to claim 66, wherein the organic solvent is selected from the list comprising ketones, carboxylic acids, hydrocarbons, cyclohydrocarbons, chlorohydrocarbons, alcohols, ethers, esters, and any combination thereof.

74. A solution according from to claim 66, wherein the organic solvent is selected from the list comprising acetone, xylene, toluene, ethanol, methylcyclohexane, ethyl acetate, diethyl ether, octane, chloroform, methylenechloride, dichloroethane, trichloroethene, tetrachloroethene, carbon tetrachloride, 1,4-dioxane, tetrahydrofuran, pyridine, triethylamine, nitromethane, acetonitrile, dimethylformamide, dimethulsulfoxide, and any combination thereof.

75. A solution according to claim 66, wherein the solution is a lyotropic liquid crystal solution or an isotropic solution.

76. A solution according to claim 66, wherein the supramolecules are formed by interaction of at least two different compounds of the general structural formula I.

77. A solution according to claim 66, wherein the supramolecules are formed by interaction of the same compounds of the general structural formula I.

78. A solution according to claim 66, further comprising surfactants.

79. A solution according to claim 66, further comprising plasticizers.

80. A compensation panel comprising at least one retardation layer being substantially transparent in the visible spectral range and comprising at least one polycyclic organic compound of a general structural formula (I), 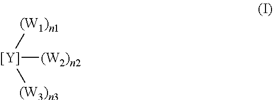
wherein Y is a predominantly planar polycyclic system being at least partially aromatic,
W₁, W₂, and W₃ are different groups providing solubility in an organic solvent, and
sum (n1+n2+n3) is 1, 2, 3, 4, 5, 6, 7 or 8,
wherein said polycyclic organic compound is capable of forming supramolecules in the organic solvent and is substantially transparent for electromagnetic radiation in the visible spectral range.

81. A compensation panel according to claim 80, wherein the polycyclic system Y is heterocyclic, and wherein the heteroatoms of the heterocyclic system Y are selected from the list comprising N, O and S.

82. A compensation panel according to claim 80, wherein the polycyclic system Y comprises at least one fragment selected from the list comprising furan, oxirane, 4H-pyran, 2H-chromene, benzo[b]furan, 2H-pyran, thiophene, benzo[b]thiophene, parathiazine, pyrrole, pyrrolidine, pyrazole, imidazole, imidazoline, imidazolidine, pyrazolidine, pyrimidine, pyridine, piperazine, piperidine, pyrazine, indole, purine, benzimidazole, quinoline, phenothiazine, morpholine, thiaziole, thiadiazole, and oxazole.

83. A compensation panel according to claim 80, wherein the polycyclic system comprises at least one fragment representing a polycyclic aromatic hydrocarbon, and wherein the polycyclic aromatic hydrocarbon is selected from the list comprising acenaphthene, acenaphthylene, acephenanthrylene, biphenylene, and naphthalene.

84. A compensation panel according to claim 80, wherein the polycyclic system Y is selected from the list comprising, oligophenyl, imidazole, pyrazole, acenaphthene, triaizine, and having a general structural formula selected from structures 1-24: 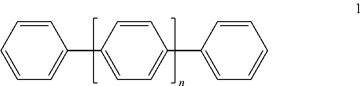
where n is the number in the range from 1 to 8

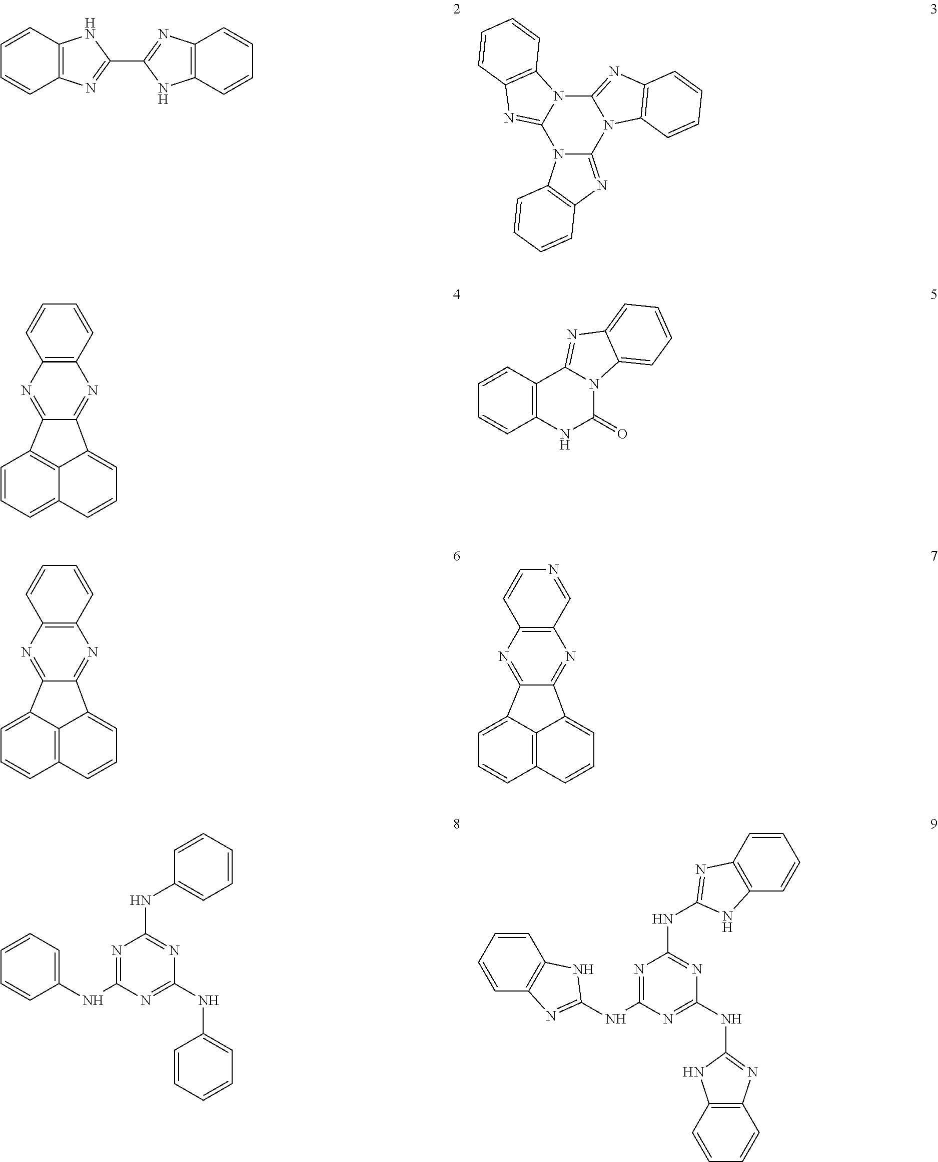

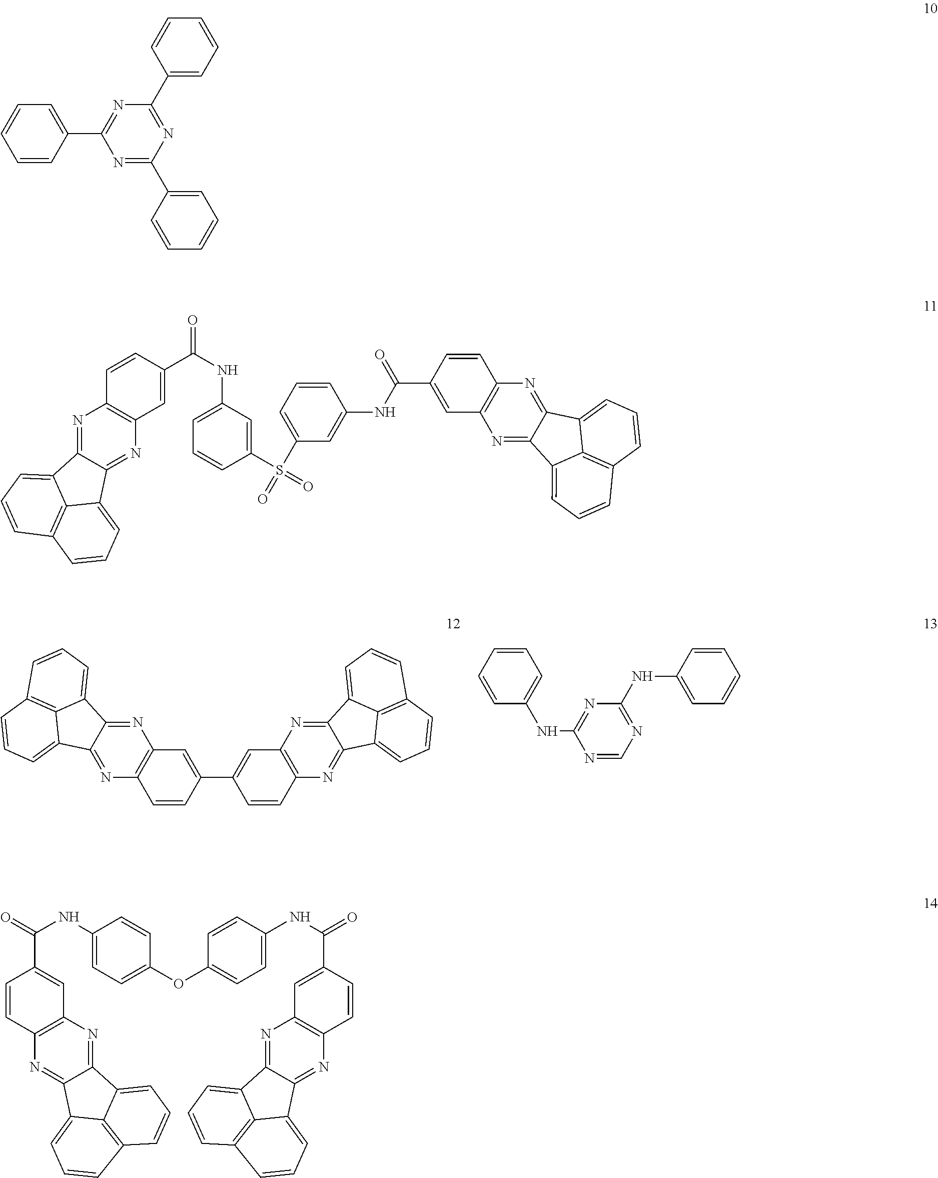

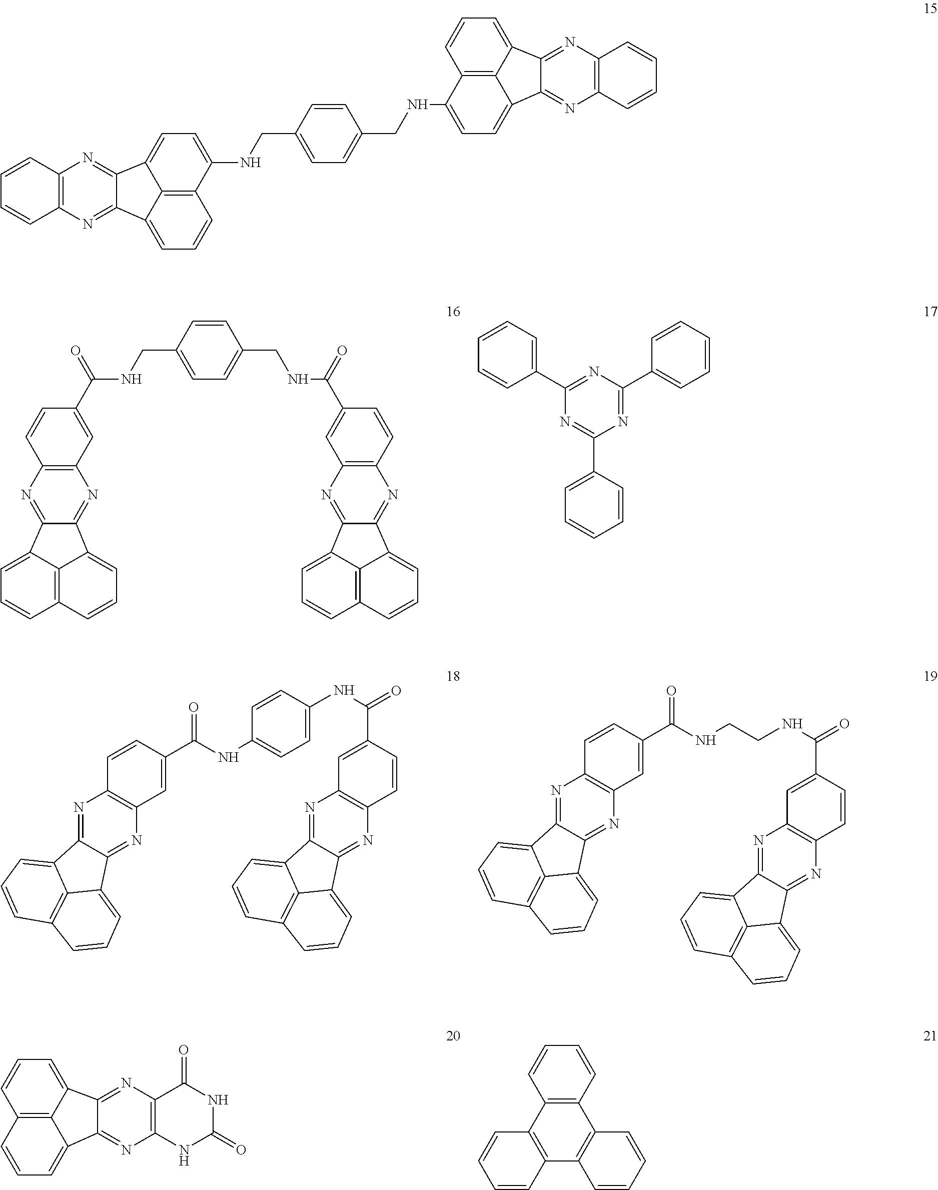

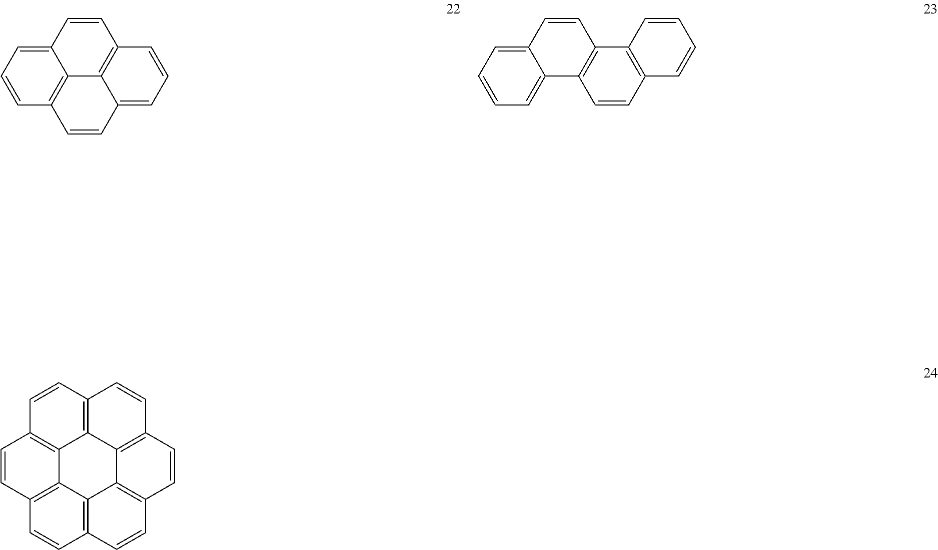

85. A compensation panel according to claim 80, in which at least one of the groups W providing solubility of the polycyclic organic compound in the organic solvent is selected from the list comprising carboxylic (COOH) group, linear and branched (C₁-C₂₀)alkyl, (C₂-C₂₀)alkenyl, and (C₂-C₂₀)alkinyl.

86. A compensation panel according to claim 80, wherein at least one of the groups W providing solubility of the polycyclic organic compound is connected with the polycyclic system Y via a bridging group A, and wherein the bridging group A of the polycyclic organic compound is selected from the list comprising —C(O)—, —C(O)O—, —C(O)—NH—, —(SO₂)NH—, —O—, —CH₂O—, —NH—, >N—, and any combination thereof.

87. A compensation panel according to claim 80, comprising two or more retardation layers, wherein at least two of said layers comprise different polycyclic compounds of the general structural formula (I).

88. A compensation panel according to claim 80, further comprising a substrate.

89. A compensation panel according to claim 88, wherein the substrate is transparent for electromagnetic radiation in the visible spectral range.

90. A compensation panel according to claim 80, wherein the substrate is made of material selected from the list comprising polymer, glass or foil.

91. A compensation panel according to claim 80, further comprising a transparent adhesive layer applied on top of the retardation layer.

92. A compensation panel according to claim 91, further comprising a protective coating applied on the adhesive transparent layer.

93. A compensation panel according to claim 80, wherein said retardation layer is at least partially crystalline.

94. A compensation panel according to claim 80, wherein the retardation layer is a biaxial retardation layer of B_{A}-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: ns>nn>nf.

95. A compensation panel according to claim 80, wherein the retardation layer is a biaxial retardation layer of A_{C}-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: ns>nf>nn.

96. A compensation panel according to claim 80, comprising at least one retardation layer of a first type having slow and fast principal axes lying substantially in the plane of the first type retardation layer, and at least one retardation layer of a second type having an optical axis directed substantially perpendicular to the plane of the second type retardation layer.

97. A compensation panel according to claim 96, wherein the retardation layer of the first type is a uniaxial retardation layer of negative A-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: nn=ns>nf.

98. A compensation panel according to claim 96, wherein the retardation layer of the first type comprises rod-like supramolecules which are oriented with their longitudinal axes substantially parallel to the fast principal axis.

99. A compensation panel according to claim 98, wherein said rod-like supramolecules have approximately isotropic polarizability in planes which are perpendicular to their longitudinal axes.

100. A compensation panel according to claim 96, wherein the retardation layer of the second type is a uniaxial retardation layer of negative C-type which is characterized by two in-plane refractive indices (nf and ns) corresponding to a fast principal axis and a slow principal axis respectively, and one refractive index (nn) in the normal direction which obey the following condition for electromagnetic radiation in the visible spectral range: nf=ns>nn.

101. A compensation panel according to claim 96, wherein the retardation layer of the second type comprises sheet-like supramolecules with their plane oriented substantially parallel to the surface of said retardation layer.
